(19) United States
(12) Patent Application Publication
(10) Pub. No.: US 2026/0242724 A1
(43) Pub. Date: Aug. 20, 2026

(54) NON-CLONING METHODS OF BOVINE EMBRYO REPLICATION

(71) Applicant: THE SEMEX ALLIANCE, Guelph (CA)

(72) Inventors: Remi Labrecque, St-Pie (CA); Si-Jung Jang, Granby (CA); Michael Lohuis, Guelph (CA); Marjorie Faust, Waunakee, WI (US)

(21) Appl. No.: 19/472,293

(22) PCT Filed: Apr. 2, 2024
(86) PCT No.: PCT/CA2024/050421

§ 371 Date: Oct. 3, 2025

Related U.S. Application Data
(60) Provisional application No. 63/456,624, filed on Apr. 3, 2023.

(51) Int. Cl. C12N 5/073

(52) U.S. Cl. CPC C12N 5/0604; C12N 2500/02; C12N 2500/36; C12N 2500/90; C12N 2501/11; C12N 2501/115; C12N 2501/15; C12N 2501/727; C12N 2506/025; C12N 2506/45; C12N 2510/00; C12N 2513/00

(57) ABSTRACT

The disclosure relates to methods, products and compositions useful for the derivation of bovine iblastoids. Methods are described that allow for naïve stem cell aggregation and iblastoid formation. Also provided are aggregation and iblastoid media suitable for promoting aggregation of naïve stem cells and preparing iblastoids. The bovine iblastoids may be used for selection and breeding programs including the replication of preimplantation embryos with desirable characteristics and in vitro breeding comprising a transgenerational breeding cycle from parental germ cells and/or embryos to offspring embryos.

### CROSS-REFERENCE TO RELATED APPLICATIONS

This application claims the benefit of priority to U.S. Provisional Application No. 63/456,624, filed Apr. 3, 2023, the contents of which are incorporated herein by reference in their entirety.

### FIELD

The present disclosure relates to the derivation of bovine embryos and more specifically to methods of generating multiple bovine embryos with desirable characteristics from a preparation of bovine naïve stem cells, as well as associated methods, selection and/or breeding programs, and compositions.

### INTRODUCTION

Widespread use of artificial insemination (AI) has been instrumental in enabling the precise genetic evaluation of sires for their use in genetic improvement programs. However, for the majority of genetic characteristics AI does not provide the capability of producing progeny with known characteristics as a result of recombination and crossing over during meiosis. Cloning technologies have been presented as solutions for providing progeny with limited genetic differences and greater uniformity. Since the 1990's, numerous cloned animals have been produced using a nuclear transfer approach. More recently when producing genetically modified livestock, researchers have used somatic cell nuclear transfer (SCNT), which many consider as the preferred technique due to the resulting genotype uniformity and lack of mosaicism (Springer et al. 2021). However, SCNT in general has resulted in low biological and economic efficiencies as a consequence of the time intensive nature of procedures such as oocyte enucleation and nuclear transfer, low embryo production rates (~20-30% of blastocyst formation), and requirements for highly skilled specialists to complete the procedure. High rates of pregnancy loss, high stillbirth rates, high incidence of calf abnormality such as large offspring syndrome and undesirable animal health and welfare are frequently associated with SCNT, most especially in bovines and other ruminants (Hill, 2014; Mueller and Van Eenennaam, 2022; Springer et al., 2021). Altogether, this severely limits the use of nuclear transfer/SCNT as a tool for producing large numbers of identical embryos for large-scale/commercial use.

In 2021, the generation of induced blastoids (iblastoids) from human pluripotent stem cells was published in Nature by two independent groups (Yu et al., 2021 and Liu et al., 2021). Both have succeeded at reassembling human blastocyst-like structures using either naïve embryonic stem cells (Yu et al., 2021) or induced pluripotent stem cells (iPSCs, Liu et al., 2021), which both showed characteristics of preimplantation embryos. Furthermore, two additional papers were published recently where the quality of human iblastoids produced was enhanced through protocol optimizations (Yanagida et al., 2021 and Kagawa et al., 2022). Human iblastoids express similar RNA transcriptome features compared to in vitro produced human blastocysts. Furthermore, iblastoids can be induced into embryonic (naïve and primed pluripotent stem cells) and extraembryonic (trophoblast stem cells and extraembryonic endodermal cells) cell lineages. Moreover, iblastoids react in a similar fashion compared to regular embryos in terms of their in vitro attachment ability when plated on endometrium cells. Based on these studies, researchers have concluded that iblastoids can be used for early human embryo development research as a replacement for real embryos. Because of ethical and regulatory issues, which limit what can be done with human stem cells and human embryos, it is still unknown whether human iblastoids will be able to develop further following transfer into a uterus.

In contrast for farm animals, Pérez-Gómez et al. (2021) conclude that “[p]rimary bovine trophectoderm cell culture can be established using relatively simple media . . . , whereas conditions required for truly pluripotent epiblast cell culture in farm animals remain to be captured.” As a result, these researchers delineated important differences in gene regulatory networks and roles in cell differentiation events through early embryonic development to embryo implantation for human, mouse, and ungulates. Also notably, placental architecture for the developing fetus differs for ruminants, horses and pigs, and human and mouse. These findings for farm animals highlight the needs for improving embryo and pluripotent cell culture and developing species-relevant assisted reproductive technologies such as the generation of non-human iblastoids.

In addition, the animal sciences can realize a broad array of benefits from iblastoid technology by enhancing genetic improvement, expanding the multiplication and distribution of desired genetic resources, improving the consistency and predictability of genetic products, reducing genetic lag, and providing a platform for research and development in embryo development, improving assisted reproductive technologies, and veterinary medical biologics and therapeutics. Important drivers of genetic change in a genetic improvement system include the accuracy and intensity of selection, genetic variation, average age of parents at the birth of offspring, and genetic lag. When genomic selection and assisted reproductive technologies such as multiple ovulation embryo transfer, ovum pick up, in vitro embryo production, and/or juvenile in vitro fertilization and embryo transfer are combined (Mueller and Van Eenennam, 2022), genetic change from animal genetic improvement programs is significantly larger. When combined, these technologies enable the accurate prediction of genetic merit early in life including for preimplantation stage embryos (Saadi et al., 2014b), shorter generation intervals, and increased selection intensity of parents. More recently, genetic modification technologies have offered new tools for making directed genetic change in animal populations (Mueller and Van Eenennam, 2022; Park, 2007; U.S. Pat. No. 7,067,713).

There is a need for new and/or improved methods for generating large numbers of identical embryos for traditional and in vitro breeding systems, large-scale and commercial use, and the production and widespread distribution of healthy, elite, and/or genetically modified livestock. The preparation of bovine iblastoids may facilitate the efficient replication of embryos with desirable characteristics. There remains a need for bovine iblastoids and associated methods for the production of iblastoids.

### SUMMARY

One of the main advantages of the iblastoids described herein is that they are produced without having to use existing cloning methods. The disclosed iblastoid production technology represents a very effective and cost-efficient method for producing large numbers of identical embryos, for generating and replicating elite genetics (small-scale use), for selection and breeding systems as described herein, and for large-scale applications in commercial settings such as the replication of genetically modified germplasm, reconstructed diploid embryos, and/or embryos with desirable characteristics.

To enable the technology for domestic animals including cattle, the 3D culture system for bovine naïve stem cells, culture conditions for producing bovine iblastoids, and selection and breeding systems for their use have been developed and evaluated as described herein.

Described herein are materials and methods useful for achieving aggregation of bovine naïve stem cells, reassembly of blastocyst-like iblastoids, as well as use of iblastoids in selection and breeding systems. As demonstrated in the Examples, bovine iblastoids can be generated from bovine naïve stem cells cultured under aggregation conditions, for example using hanging drop culture, low attachment dishes, or microwell devices. As shown herein, naïve stem cells plated in a microwell device provide greater aggregation and uniformity in size of aggregates compared to cells aggregated using hanging drop or low attachment dish methods. Furthermore, iblastoid media compositions and associated methods are described which support iblastoid formation, allowing for the preparation of bovine iblastoids. The embodiments described herein are therefore useful for preparing bovine iblastoids and optionally for use in selection and breeding programs such as for the replication of preimplantation embryos with desirable genetic and epigenetic characteristics. Accordingly, in one aspect there is provided a method for preparing bovine iblastoids the method comprising:
a) providing a population of bovine naïve stem cells;
b) culturing the bovine naïve stem cells in aggregation media under aggregation conditions to produce aggregated bovine naïve stem cells;
c) introducing a first iblastoid media, optionally wherein the aggregation media and non-aggregated cells are removed before introducing the first iblastoid media;
d) culturing the aggregated bovine naïve stem cells in the first iblastoid media under hypoxic conditions;
e) introducing a second iblastoid media, optionally wherein the first iblastoid media is removed before introducing the second iblastoid media; and
f) culturing the aggregated bovine naïve stem cells in the second iblastoid media under hypoxic conditions to obtain the bovine iblastoids.

In an embodiment, the population of bovine naïve stem cells in step a) are dissociated bovine naïve stem cells and/or the bovine naïve stem cells are bovine embryo-derived naïve stem cells.

In an embodiment, step b) comprises introducing the cells into one or more wells of a microwell device and culturing the cells for about 24 hours. In an embodiment, the wells of the microwell device have a diameter of about 400 μm to about 600 μm. In an embodiment, the population of bovine naïve stem cells is added to the microwell device at a concentration of approximately 20-60 cells/well, optionally at a concentration of approximately 20-30 cells/well.

In an embodiment, the cells are cultured in step d) and/or step f) for about 48 hours.

In an embodiment, the aggregation media comprises: a serum replacement component; and a Rho-associated, coiled-coil containing protein kinase (ROCK) inhibitor component. In an embodiment, the serum replacement component comprises a) an N2B27 component, optionally about 1% B27 component and about 0.5% N2 component; b) an insulin-transferrin-selenium component, optionally insulin-transferrin-selenium-ethanolamine (ITS-X) or insulin-transferrin-selenium (ITS-G); c) serum replacement (SR); or d) knock-out serum replacement (KOSR); and/or the ROCK inhibitor component comprises Y27632, fasudil, Thiazovivin, or Blebbistatin, optionally 5-10 μM Y27632.

In an embodiment, the first iblastoid media comprises: a serum replacement component; a MEK/ERK inhibitor component; a TGFβ-1 inhibitor component; a HIPPO pathway inhibitor component; and a ROCK inhibitor component. In an embodiment, the serum replacement component comprises a) an N2B27 component, optionally about 1% B27 supplement and about 0.5% N2 supplement; b) an insulin-transferrin-selenium component, optionally insulin-transferrin-selenium-ethanolamine (ITS-X) or insulin-transferrin-selenium (ITS-G); c) serum replacement (SR); or d) knock-out serum replacement (KOSR). In an embodiment, the MEK/ERK inhibitor component comprises PD0325901, Ravoxertinib, GSK1120212, MEK162, PD184352, Trametinib, LY3214996, or Ulixertinib, optionally about 1 μM PD0325901. In an embodiment, the TGFβ-1 inhibitor component comprises A83-01, SB431542, LY2109761, SB525334, SB505124, LDN-193189, RepSox, BIBF-0775, TGFβRI-IN-3, or TP0427736, optionally about 1 μM A83-01. In an embodiment, the HIPPO pathway inhibitor component comprises lysophosphatidic acid (LPA), optionally about 1 μM LPA. In an embodiment, the ROCK inhibitor component comprises Y27632, fasudil, Thiazovivin, or Blebbistatin, optionally 5-10 μM Y27632.

In an embodiment, the first iblastoid media further comprises a second serum replacement component, optionally ITS-X, optionally 0.1-1% ITS-X; a LIF component, optionally human LIF, bovine LIF, caprine LIF, porcine LIF, buffalo LIF, or recombinant LIF, optionally at a concentration of about 10 ng/ml; an epidermal growth factor (EGF) component, optionally about 50 ng/ml EGF; a Wnt agonist, optionally Wnt3a, Wnt agonist 1, or SKL2001, optionally 10-100 ng/mL Wnt3a; a GSK33 inhibitor, optionally CHIR99021, BIO, CHIR-98014, LY2090314, or IM-12, optionally 1-2 μM CHIR99021; a histone deacetylase inhibitor, optionally valproic acid (VPA), sodium butyrate, or trichostatin A, optionally 0.2-0.8 mM VPA; and/or a second TGFβ-1 inhibitor, optionally 0.1-1 μM SB431542. In embodiments where the first iblastoid media comprises an EGF component, the first iblastoid media may further comprise an fibroblast growth factor (FGF) component, optionally basic fibroblast growth factor (bFGF) or FGF4, optionally at a concentration of 10-30 ng/ml.

In an embodiment, the second iblastoid media comprises: a serum replacement component; a TGFβ-1 inhibitor component; a HIPPO pathway inhibitor component; and a ROCK inhibitor component. In an embodiment, the serum replacement component comprises a) an N2B27 component, optionally about 1% B27 supplement and about 0.5% N2 supplement; b) an insulin-transferrin-selenium component, optionally insulin-transferrin-selenium-ethanolamine (ITS-X) or insulin-transferrin-selenium (ITS-G); c) serum replacement (SR); or d) knock-out serum replacement (KOSR). In an embodiment, the TGFβ-1 inhibitor component comprises A83-01, SB431542, LY2109761, SB525334, SB505124, LDN-193189, RepSox, BIBF-0775, TGFβRI-IN-3, or TP0427736, optionally about 1 μM A83-01. In an embodiment, the HIPPO pathway inhibitor component comprises lysophosphatidic acid (LPA), optionally about 1 μM LPA. In an embodiment, the ROCK inhibitor component comprises Y27632, fasudil, Thiazovivin, or Blebbistatin, optionally 5-10 μM Y27632.

In an embodiment, the second iblastoid media further comprises one or more of a second serum replacement component, optionally ITS-X, optionally 0.1-1% ITS-X; an fibroblast growth factor (FGF) component, optionally basic fibroblast growth factor (bFGF) or FGF4, optionally at a concentration of 10-30 ng/ml; an Activin A component, optionally human Activin A, bovine Activin A, or murine Activin A, optionally at a concentration of 5-50 ng/ml; and/or a GSK33 inhibitor, optionally CHIR99021, BIO, CHIR-98014, LY2090314, or IM-12, optionally 1-3 μM CHIR99021. In embodiments where the second iblastoid media comprises an FGF component, the second iblastoid media may further comprise an epidermal growth factor (EGF) component, optionally about 50 ng/ml EGF.

In an embodiment, the hypoxic conditions comprise about 5%-10% CO₂, optionally about 6.8% CO₂, and about 1%-5% O₂, optionally about 5% O₂.

In an embodiment, the first iblastoid media and/or the second iblastoid media is replaced with fresh media after about 24 hours.

In one embodiment, the population of bovine naïve stem cells are derived from primed pluripotent stem cells or induced pluripotent stem cells.

In one embodiment, the population of bovine naïve stem cells are embryo-derived naïve stem cells, optionally derived from a preimplantation embryo or embryo-derived expanded pluripotent stem cells.

In one embodiment, the population of bovine naïve stem cells are derived by a method comprising:
providing a bovine embryo comprising bovine naïve stem cell-like cells, optionally the bovine embryo is Zona Pellucida (ZP)-free;
contacting the bovine embryo with an extracellular matrix (ECM)-coated substrate, wherein the ECM-coated substrate comprises a substrate comprising a negatively charged substrate surface adjacent to a positively charged biocompatible polymer layer, and a negatively charged ECM layer adjacent to the positively charged biocompatible polymer layer; and
culturing the bovine embryo in the presence of outgrowth medium to induce attachment of the bovine embryo to the ECM-coated substrate and outgrowth of an inner cell mass (ICM) comprising derived bovine naïve stem cells.

In an embodiment, the bovine naïve stem cells are genetically modified and/or genome edited cells.

In an embodiment, the method further comprises performing testing for one or more biomarkers to determine one or more characteristics of the population of bovine naïve stem cells for one or more biomarkers, and optionally determining a score based on the determined characteristics.

In an embodiment, the method further comprises selecting the population of bovine naïve stem cells based on the one or more characteristics determined by testing for one or more biomarkers and/or the score.

In an embodiment, the method further comprises performing testing for one or more biomarkers to determine one or more characteristics of the iblastoids, and optionally determining a score based on the determined characteristics.

In an embodiment, the method further comprises selecting one or more of the iblastoids based on the one or more characteristics determined by testing for one or more biomarkers and/or the score.

An aspect includes a plurality of iblastoids prepared using the methods described herein.

A further aspect includes use of the iblastoid technology described herein for selection and breeding systems. Accordingly, provided herein is a method of cattle breeding, the method comprising:
a) selecting at least one bovine parent from a plurality of animals;
b) obtaining a plurality of gametes from the at least one bovine parent;
c) creating at least one embryo from the plurality of gametes;
d) deriving a population of naïve stem cells from the at least one embryo;
e) preparing one or more iblastoids according to the methods described herein; and
f) selecting the one or more iblastoids for breeding, optionally in vitro breeding, and/or for transfer to recipient female surrogates for producing offspring.

In an embodiment, the at least one embryo is tested for one or more biomarkers to determine one or more characteristics of the at least one embryo, and optionally a score is determined based on the determined characteristics.

Also provided herein is an aggregation media composition comprising: a serum replacement component; and a Rho-associated, coiled-coil containing protein kinase (ROCK) inhibitor component. In an embodiment, the serum replacement component comprises a) an N2B27 component, optionally about 1% B27 component and about 0.5% N2 component; b) an insulin-transferrin-selenium component, optionally insulin-transferrin-selenium-ethanolamine (ITS-X) or insulin-transferrin-selenium (ITS-G); c) serum replacement (SR); or d) knock-out serum replacement (KOSR); and/or the ROCK inhibitor component comprises Y27632, fasudil, Thiazovivin, or Blebbistatin, optionally 5-10 μM Y27632.

Also provided herein is an iblastoid media 1 composition comprising: a serum replacement component; a MEK/ERK inhibitor component; a TGFβ-1 inhibitor component; a HIPPO pathway inhibitor component; and a ROCK inhibitor component. In an embodiment, the serum replacement component comprises a) an N2B27 component, optionally about 1% B27 component and about 0.5% N2 component; b) an insulin-transferrin-selenium component, optionally insulin-transferrin-selenium-ethanolamine (ITS-X) or insulin-transferrin-selenium (ITS-G); c) serum replacement (SR); or d) knock-out serum replacement (KOSR). In an embodiment, the MEK/ERK inhibitor component comprises PD0325901, Ravoxertinib, GSK1120212, MEK162, PD184352, Trametinib, LY3214996, or Ulixertinib, optionally about 1 μM PD0325901. In an embodiment, the TGFβ-1 inhibitor component comprises A83-01, SB431542, LY2109761, SB525334, SB505124, LDN-193189, RepSox, BIBF-0775, TGFβRI-IN-3, or TP0427736, optionally about 1 μM A83-01. In an embodiment, the HIPPO pathway inhibitor component comprises lysophosphatidic acid (LPA), optionally about 1 μM LPA. In an embodiment, the ROCK inhibitor component comprises Y27632, fasudil, Thiazovivin, or Blebbistatin, optionally 5-10 μM Y27632. In an embodiment, the iblastoid media 1 composition further comprises one or more of: a second serum replacement component, optionally 0.1-1% ITS-X; a LIF component, optionally human LIF, bovine LIF, caprine LIF, porcine LIF, buffalo LIF, or recombinant LIF, optionally at a concentration of about 10 ng/ml; an epidermal growth factor (EGF) component, optionally about 50 ng/ml EGF; a GSK33 inhibitor, optionally CHIR99021, BIO, CHIR-98014, LY2090314, or IM-12, optionally 1-2 μM CHIR99021; a Wnt activator, optionally Wnt3a, Wnt agonist 1, or SKL2001, optionally 10-100 ng/mL Wnt3a; a histone deacetylase inhibitor, optionally valproic acid (VPA), sodium butyrate, or trichostatin A, optionally 0.2-0.8 mM VPA; and/or a second TGFβ-1 inhibitor component, optionally 0.1-1 μM SB431542. In embodiments where the first iblastoid media comprises an EGF component, the first iblastoid media may further comprise an fibroblast growth factor (FGF) component, optionally basic fibroblast growth factor (bFGF) or FGF4, optionally at a concentration of 10-30 ng/ml.

Also provided herein is an iblastoid media 2 composition comprising: a serum replacement component; a TGFβ-1 inhibitor component; a HIPPO pathway inhibitor component; and a ROCK inhibitor component. In an embodiment, the serum replacement component comprises a) an N2B27 component, optionally about 1% B27 component and about 0.5% N2 component; b) an insulin-transferrin-selenium component, optionally insulin-transferrin-selenium-ethanolamine (ITS-X) or insulin-transferrin-selenium (ITS-G); c) serum replacement (SR): or d) knock-out serum replacement (KOSR). In an embodiment, the TGFβ-1 inhibitor component comprises A83-01, SB431542, LY2109761, SB525334, SB505124, LDN-193189, RepSox, BIBF-0775, TGFβRI-IN-3, or TP0427736, optionally about 1 μM A83-01. In an embodiment, the HIPPO pathway inhibitor component comprises lysophosphatidic acid (LPA), optionally about 1 μM LPA. In an embodiment, the ROCK inhibitor component comprises Y27632, fasudil, Thiazovivin, or Blebbistatin. In an embodiment, the iblastoid media 2 composition further comprises one or more of: a second serum replacement component, optionally ITS-X, optionally 0.1-1% ITS-X; an fibroblast growth factor (FGF) component, optionally basic fibroblast growth factor (bFGF) or FGF4, optionally at a concentration of 10-30 ng/ml; an Activin A component, optionally human Activin A, bovine Activin A, or murine Activin A, optionally at a concentration of 5-50 ng/ml; a Wnt activator, optionally Wnt3a, Wnt agonist 1, or SKL2001; and/or a GSK33 inhibitor, optionally CHIR99021, BIO, CHIR-98014, LY2090314, or IM-12, optionally 1-3 μM CHIR99021. In embodiments where the iblastoid media 2 comprises an FGF component, the iblastoid media 2 may further comprise an epidermal growth factor (EGF) component, optionally about 50 ng/ml EGF.

A further aspect includes use of a media composition described herein for preparing bovine iblastoids.

The preceding section is provided by way of example only and is not intended to be limiting on the scope of the present disclosure and appended claims. Additional objects and advantages associated with the compositions and methods of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages, objects, and embodiments are expressly included within the scope of the present disclosure. The publications and other materials used herein to illuminate the background of the disclosure, and in particular cases, to provide additional details respecting the practice, are incorporated by reference, and for convenience are listed in the appended reference section.

### DRAWINGS

Further objects, features and advantages of the disclosure will become apparent from the following detailed description taken in conjunction with the accompanying figures showing illustrative embodiments of the disclosure, in which:

FIG. 1 shows Trophoblast Stem (TS) cell organoids generation using the hanging drop method. A: Hanging drop dish (left) and stereomicroscopic image showing the cell suspension (right). B: Creating perfect drops can be challenging (arrows indicate broken drops.) C: Cell aggregation after 48 h. Organoids have similar shapes as regular embryos with a blastocoel cavity (arrow), or have the appearance of collapsed embryos (star). D: Bright field (left) and DNA staining (right) of TS organoid. E: DNA staining of day-7 blastocyst (dotted circle indicates ICM).

FIG. 2 shows images of Trophoblast Stem (TS) cell-derived blastocyst-like structures generated by low attachment dish. Small cell clusters at Day 0 have grown into blastocyst-like structures after 48 hrs.

FIG. 3 shows a microwell device (Stemfit3D, Cat #H853400) inserted in a 35 mm dish (left) and an image of the microwell device under stereomicroscope (right).

FIG. 4 shows images of blastocyst-like structures generated using a microwell system. A: Trophoblast Stem (TS) cell aggregates in the microwell after 24 hours. B: Image of several TS cell-derived blastocyst-like structures (after transfer to a standard culture dish) after 48 hrs of culture in the microwell. C: Bovine naïve stem cells derived aggregates in microwell after 24 hours. D: Bovine naïve stem cells derived aggregates in microwell after 48 hours. E: Image of blastocyst-like structure showing blastocoel cavity (red arrow) derived from bovine naïve stem cells after 96 hours of culture in the microwell.

FIG. 5 shows SOX2/CDX2/SOX17 (S/C/S) staining of blastocysts (day-7, day-11 embryos) and day-5 iblastoids for inner cell mass (ICM), trophectoderm (TE) and hypoblast markers. SOX2 (ICM), CDX2 (TE) and SOX17 (hypoblast) positive cells are well organized through the structure of the embryo according to the developmental stage. B: SOX2 (ICM), CDX2 (TE) positive cells are well organized through the structure of the iblastoid. SOX2⁺/CDX2⁻ regions indicate the structure of the cavity (arrowhead).

FIG. 6 shows a schematic of steps involved in generating multiple genetically identical embryos (also called iblastoids) from bovine naïve stem cells.

FIG. 7 shows steps for characterizing candidate germplasm for its suitability for target markets. Candidate germplasm meeting specified requirements can be used in selection and breeding systems such as those shown in FIGS. 8 and 9.

FIG. 8 shows a schematic of a selection and breeding system using iblastoids for the production of crossbred animals for target commercial markets with specific market requirements.

FIG. 9 shows a schematic of a selection and breeding system using iblastoids for the production of purebred animals for target commercial markets with specific market requirements.

### DESCRIPTION OF VARIOUS EMBODIMENTS

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature described herein may be combined with any other feature or features described herein.

### I. General Definitions

As used herein, the following terms may have meanings ascribed to them below, unless specified otherwise. However, it should be understood that other meanings that are known or understood by those having ordinary skill in the art are also possible, and within the scope of the present disclosure. All publications, patent applications, patents, and other references mentioned herein are incorporated by reference in their entirety. In the case of conflict, the present specification, including definitions, will control. In addition, the materials, methods, and examples are illustrative only and not intended to be limiting.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges are also encompassed within the description, subject to any specifically excluded limit in the stated range. Where the stated range includes one or both of the limits, ranges excluding either or both of those included limits are also included in the description.

It must be noted that as used herein and in the appended claims, the singular forms “a”, “an”, and “the” include plural references unless the context clearly dictates otherwise.

All numerical values within the detailed description and the claims herein are modified by “about” or “approximately” the indicated value, and take into account experimental error and variations that would be expected by a person having ordinary skill in the art.

The phrase “and/or”, as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either”, “one of”, “only one of”, or “exactly one of.”

In the claims, as well as in the specification above, all transitional phrases such as “comprising”, “including”, “carrying”, “having”, “containing”, “involving”, “holding”, “composed of”, and the like are to be understood to be open-ended, i.e., to mean including but not limited to. Only the transitional phrases “consisting of” and “consisting essentially of” shall be closed or semi-closed transitional phrases, respectively.

As used herein in the specification and in the claims, the phrase “at least one”, in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The term “about” as used herein means plus or minus 10%-15%, 5-10%, or optionally about 5% of the number to which reference is being made.

It should be understood that, in certain methods described herein that include more than one step or act, the order of the steps or acts of the method is not necessarily limited to the order in which the steps or acts of the method are recited unless the context indicates otherwise.

It should also be understood that any methods and materials similar or equivalent to those described herein can also be used in the practice or testing of the present disclosure.

### II. Methods

Described herein are methods for preparing bovine iblastoids. As set out in the Examples, the inventors have demonstrated the formation of bovine iblastoids having a blastocyst-like structure, including a blastocoel cavity and comprising cells expressing markers of the ICM (express SOX2), and TE (express CDX2). Following further culture, the iblastoids comprise hypoblast cells (express SOX17). The materials and methods described herein are therefore useful for deriving and maintaining bovine iblastoids, and optionally for use in breeding programs such as for the replication of preimplantation embryos with desirable characteristics.

Accordingly, in one aspect there is provided a method for preparing bovine iblastoids. In one embodiment, the method comprises:
a) providing a population of bovine naïve stem cells;
b) culturing the bovine naïve stem cells in aggregation media under aggregation conditions to produce aggregated bovine naïve stem cells;
c) introducing a first iblastoid media, optionally, wherein the aggregation media and non-aggregated cells are removed before introducing the first iblastoid media;
d) culturing the aggregated bovine naïve stem cells in the first iblastoid media under hypoxic conditions, optionally for about 48 hours;
e) introducing a second iblastoid media, optionally wherein the first iblastoid media is removed before introducing the second iblastoid media; and
f) culturing the aggregated bovine naïve stem cells in the second iblastoid media under hypoxic conditions, optionally for about 48 hours, to obtain the bovine iblastoids.

As used herein, the term “iblastoid” refers to a spheric 3D structure having a diameter between 100-200 μm (closely matching the size of a day-7 blastocyst), and a blastocyst-like structure comprising 2 morphologically distinct cell lineages, namely inner cell mass (ICM) and trophectoderm (TE) cells, and a cavity. When appropriately stained for example at about 5 to 7 days after aggregation has been initiated, the iblastoid is marked by appropriate S/C/S staining, namely a clear SOX2 staining for the ICM cells and CDX2 for TE cells, but no SOX17 signal (resembling day-7 embryos). However, using extended in vitro embryo culture and staining, SOX17 signal may become visible as early as about 7-9 days after aggregation has been initiated (resembling day-9 embryos). The iblastoid can be used to derive naïve stem cells, primed stem cells and trophoblast stem cells when the iblastoid is subjected to the specific conditions for each type of cell, or may be transferred into a recipient or surrogate for further growth and development.

As used herein, the term “naïve stem cell” refers to stem cells which are capable of being derived and maintained in an undifferentiated state of self-renewal without the need for exogenously expressed pluripotency factors and are capable of developing into a complete organism and/or may retain the capacity to give rise to the full complement of extraembryonic tissues, adult tissues, and/or cell types. Naïve stem cells exhibit molecular characteristics that are substantially similar to morula and ICM cells of early blastocysts and female naïve stem cells have an X chromosome signature similar to preimplantation embryos. Bovine naïve stem cells may be identified for example by round, dome-shaped cell colony morphology, and co-expression of one or more pluripotency markers such as SOX2, OCT4, or NANOG, and one or more of the naïve factors SUSD2 and/or TFCP2L1 and/or KLF4 and/or other selected pluripotency and naïve-specific factors such as those described by Messmer et al. (2019). It is understood that naïve stem cells are unlikely to exist in vivo, but can be derived from sufficiently undifferentiated ICM cells, referred to herein as “naïve stem cell-like cells”, under certain cell culture conditions.

Naïve stem cells, such as bovine naïve stem cells, may be derived directly from sufficiently undifferentiated tissues, such as for example a blastomere (2-4 cell embryo), or the cells of the ICM of a preimplantation embryo, for example a morula (stage 4); a blastocyst (stage 5); an expanding blastocyst (stage 6); an expanded blastocyst (stage 7); a hatching blastocyst (stage 8) or a hatched blastocyst (stage 9). In one embodiment, bovine naïve stem cells may be derived from a 2- to 8-day bovine embryo, optionally a 2-, 3-, 4-, 5-, 6-, 7-, or 8-day bovine embryo, or a 5- to 7-day bovine embryo. In an embodiment, the bovine embryo is a blastomere (2-4 cell embryo); a morula (stage 4); a blastocyst (stage 5); an expanding blastocyst (stage 6); an expanded blastocyst (stage 7); a hatching blastocyst (stage 8) or a hatched blastocyst (stage 9). In an embodiment, the bovine embryo is a 3- to 7-day embryo, optionally a 5- to 7-day embryo or 6- or 7-day embryo. In an embodiment, the embryo-derived cells are ICM-derived cells. In an embodiment, the bovine embryo is a Zona Pellucida-free bovine embryo. In one embodiment, the embryo is a preimplantation embryo. In one embodiment, the embryo is an embryo that has previously been frozen and/or biopsied.

Naïve stem cells, such as bovine naïve stem cells, may be derived for example using the methods as described in Patent Cooperation Treaty Application No. PCT/CA2022/051664, filed Nov. 11, 2022, or methods for expanded pluripotent stem cells as described by Zhao et al. (2021). In an embodiment, bovine naïve stem cells are embryo-derived naïve stem cells. In an embodiment, bovine naïve stem cells are embryo-derived expanded pluripotent stem cells. In one embodiment, the embryo has been selected based on testing for one or more biomarkers.

The resetting of cell potency is recognized as an alternative method for deriving naïve stem cells. For example, naïve stem cells, such as bovine naïve stem cells, may be derived from primed embryonic stem cells as is described by Guo et al. (2017) or from induced pluripotent stem cells (iPSC) which may be derived from fully differentiated cells such as fibroblasts as disclosed in Liu et al. (2021) or WO 2022/109667 A1 (Methods and Cellular Structures). Accordingly, in an embodiment, bovine naïve stem cells are iPSC-derived naïve pluripotent stem cells. In another embodiment, bovine naïve stem cells are derived from primed pluripotent stem cells. Sources of pluripotent stem cells (e.g. primed, expanded, or induced pluripotent stem cells) may include those derived using methods described, for example, in Soto et al. (2021), Bigliotti et al. (2018), Zhao et al. (2021), or Han et al. (2011).

Naïve stem cells can also be derived from in vitro produced embryos using methods known in the art. For example, oocytes and sperm may be collected from a mating pair (dam and sire) and used to produce an embryo via in vitro fertilization techniques. In an embodiment, the dam and/or sire are tested for one or more biomarkers, and optionally scored and/or selected for in vitro fertilization based on the results of said testing.

Alternatively, naïve stem cells can be derived from diploid reconstructed biparental embryos. Diploid embryos with predetermined genomes can be generated in vitro by reconstructing biparental embryos using screened and selected androgenetic and parthenogenetic embryonic haploid cells, for example as described in WO 2020/168422 (Use of Haploid Embryonic Cells to Generate Offspring with Predetermined Genomes), the contents of which is incorporated by reference herein in its entirety. Accordingly, in one embodiment, the bovine embryo is a reconstructed diploid embryo. Genomes for the reconstructed diploid embryos can be produced to contain a unique combination of alleles, haplotypes, epigenetic marks, or traits meeting stringent genetic and/or predicted performance ability criteria.

In various embodiments described herein, the naïve stem cells are dissociated prior to culturing under aggregation conditions. Various methods can be used to dissociate the naïve stem cells, for example as described in Example 1. Other methods may also be used.

In various embodiments described herein, the naïve stem cells have been tested for one or more biomarkers, and optionally scored and/or selected based on testing for one or more biomarkers.

As used herein, “testing for one or more biomarkers” includes testing for genetic, genomic, and/or epigenetic characteristics, including, but not limited to, the presence or absence of one or more specific alleles, single nucleotide polymorphisms (SNPs), genomic insertions and/or deletions, histone modifications, protamine modifications, DNA methylation, gene or mRNA expression levels, protein expression or modifications, and metabolite analysis. Testing for one or more biomarkers may be carried out at any step of the methods described herein. For example, testing may be carried out on embryos, outgrowths, stem cells, iblastoids, sperm, oocytes, and/or animals (individual dams and sires, or optionally a mating pair).

As used herein, a “trait” or “characteristic” refers to a specific feature of an animal, embryo, outgrowth, stem cell, or iblastoid which may be influenced or determined by one or more genetic factors (e.g. allelic variants, epigenetic marks, and combinations thereof) and/or environmental factors.

In some embodiments, the naïve stem cells have been genetically modified.

A “genetically modified cell” refers to a cell where genomic DNA of the cell has been manipulated to express one or more exogenous genes and/or to introduce mutation(s) within endogenous genes or intergenic regions which affects expression or functional activity of one or more endogenous genes or gene products. Examples of successful genetic modifications in livestock have included the introduction of transgenes by microinjection (U.S. Pat. No. 7,067,713) and lentiviral infection (Park, 2007) and most recently genome editing (reviewed by Bishop and Van Eenennaam, 2020 for livestock and by Mueller and Van Eenennaam, 2022 for cattle) using transfection and genome editors, such as Zinc Finger Nucleases, transcription activator like effector nuclease (TALEN), and clustered regularly interspaced short palindromic repeat/CRISPR associated gene (CRISPR/Cas) system. Common bovine targets for genetic modification are, for example, milk protein genes such as β-lactoglobulin, β-casein, myostatin, horned/polled, prolactin receptor conferring a slick haircoat for improving heat tolerance, and various genes involved in disease susceptibility or resilience (Bishop and Van Eenennam, 2020 and Mueller and Van Eenennaam, 2022).

Likewise, a “genetically modified embryo” refers to an embryo where the genomic DNA of cells in the embryo has been manipulated to express one or more exogenous genes and/or to introduce mutation(s) within endogenous genes or intergenic regions which affects expression or functional activity of one or more endogenous genes or gene products.

Populations of dissociated bovine naïve stem cells may be obtained by dissociating a population of bovine naïve stem cells, for example using the methods described herein or other methods known in the art.

The term “aggregation conditions” refers to conditions which promote the formation of cell aggregates. For example, as shown in the Examples herein, cells can be aggregated using a hanging drop method in which small drops of dissociated cells are suspended from a surface such as the underside of the lid of a tissue culture dish, and the cells come into contact and adhere to one another after settling to the bottom of the drop. Accordingly, in an embodiment, the aggregation conditions comprise a hanging drop. In another Example shown herein, cells can be aggregated using low attachment culture conditions, in which cells are introduced into an environment (such as a low attachment dish) in which cellular attachment to surfaces in the environment is inhibited and the cells adhere instead to one another. Accordingly, in an embodiment, the aggregation conditions comprise a low-attachment environment, optionally an untreated, low-attachment, or non-adherent dish or plate. In a further Example shown herein, culturing cells in a non-adherent or ultra-low attachment microwell device results in the formation of relatively homogeneous blastocyst-like structures.

As used herein, the term “microwell device” refers to a container, tube, cell culture dish, or cell culture plate containing one or more culture spaces (e.g. wells) having a diameter in the micrometer range. For example, a microwell device can include one or more wells of diameter ranging from about 100 μm to about 1000 μm, optionally from about 400 μm to about 600 μm. The microwell device may comprise any number of culture spaces (or wells) from 1 well to about 1000 or more wells. Standard configurations may comprise 6-, 12-, 24-, 48-, or 96-well plates or inserts. The microwell device may comprise a non-adherent, non-coated, or ultra-low attachment material, for example untreated or ultra-low attachment treated plastic or a non-plastic material such as silicon. The microwell device may have a rounded- or conical-shaped bottom to induce efficient cell aggregation. Commercially available microwell devices which may be used in the methods described herein include, without limitation, Stemfit3D (Microfit, Cat #; H389600L, H389600H, H853400 or H1613200); Aggrewell (Stemcell technology, Cat #34411, 34421, 34811 or 34821); non-adherent 96-well plate (Thermo Scientific™, Cat #174925, 174927; Corning, Cat #CLS7007). Customized non-adherent microwell devices, optionally having a well diameter ranging from 400 μm to about 600 μm, may also be used. In one embodiment, a microwell device can comprise a plastic/silicon based biocompatible polymer insert.

As understood in the art, the terms “incubate” or “incubating” means to maintain for example a substance, material, composition, etc. at a particular temperature, or within a temperature range, for a period of time.

As used herein, the term “physiological pH” means a pH of about 7.1 to about 7.6, optionally about 7.15 to about 7.45, about 7.2 to about 7.4, about 7.25 to about 7.35, or about 7.3.

As understood in the art, pH is influenced by the carbon dioxide (CO₂) concentration in the environment. Typical concentrations used for tissue culture range from about 5% CO₂ to about 10% CO₂, optionally about 5% CO₂ or about 6.8% CO₂.

As used herein, the term “hypoxic conditions” means conditions with an oxygen (O₂) concentration lower than atmospheric oxygen concentrations, namely lower than about 20.95% oxygen. For example, when cells are incubated under hypoxic conditions, the cells are incubated in a reduced oxygen environment, for example between about 1% and about 15% oxygen, optionally between about 1% and 10% oxygen, or between about 1% and about 5% oxygen.

As shown herein, bovine naïve stem cells are aggregated and form iblastoid structures by sequential culture in various media compositions, including aggregation media, iblastoid media 1, and iblastoid media 2. The various media may comprise for example a base medium, and one or more small molecules, growth factors, and/or nutrients. Suitable base media can be readily determined by the skilled person and includes without limitation DMEM/F12 (Dulbecco's Modified Eagle's Medium F12), advanced DMEM/F12 and Neurobasal medium. Suitable supplements can be readily determined by the skilled person and includes without limitation Minimum Essential Medium (MEM), non-essential amino acids (NEAA), L-glutamine (e.g. 1-2 mM), Glutamax, ascorbic acid, insulin, BSA (fraction V), beta-mercaptoethanol, penicillin/streptomycin, and/or gentamycin. In an embodiment, the base medium comprises 1:1 DMEM/F12 and Neurobasal medium, 1× Glutamax, 1×NEAA, 0.1 mM β-mercaptoethanol, and 50 IU/ml Pen/Strep.

In one aspect of the disclosure there are provided aggregation media components useful for deriving and maintaining iblastoids. In one embodiment, the aggregation media comprises a base medium and/or supplements as well as one or more aggregation medium components. In an embodiment, the aggregation medium comprises one or more of a serum replacement component; and a ROCK inhibitor component. In an embodiment, the serum replacement component comprises one or more of a) an N2B27 component comprising B27 supplement and N2 supplement, b) an insulin-transferrin-selenium component, optionally insulin-transferrin-selenium-ethanolamine (ITS-X) or insulin-transferrin-selenium (ITS-G); c) serum replacement (SR; Sigma), or d) knock-out serum replacement (KOSR; Gibco). In an embodiment, the N2B27 component comprises about 0.5% to about 2% B27 supplement, optionally about 1% B27 supplement, and about 0.5% to about 1% N2 supplement, optionally about 0.5% N2 supplement. In an embodiment, the ROCK inhibitor component comprises Y27632, fasudil, Thiazovivin, or Blebbistatin. In an embodiment, the aggregation medium may comprise N2B27 and Y27632.

In one aspect of the disclosure there are provided iblastoid media components useful for deriving and/or maintaining iblastoids.

In one embodiment, a first iblastoid medium comprises a base medium and/or supplements as well as one or more first iblastoid medium components. In an embodiment, the first iblastoid medium (also referred to herein as iblastoid media 1) comprises a serum replacement component, optionally an N2B27 component comprising B27 supplement and N2 supplement, optionally comprising about 0.5% to about 2% B27 supplement, optionally about 1% B27 supplement, and about 0.5% to about 1% N2 supplement, optionally about 0.5% N2 supplement; a MEK/ERK inhibitor component, optionally PD0325901, Ravoxertinib, GSK1120212, MEK162, PD184352, Trametinib, LY3214996, or Ulixertinib; a TGFβ-1 inhibitor, optionally A83-01, SB431542, LY2109761, SB525334, SB505124, LDN-193189, RepSox, BIBF-0775, TGFβRI-IN-3, or TP0427736; a HIPPO pathway inhibitor, optionally lysophosphatidic acid (LPA); and a ROCK inhibitor component, optionally Y27632, fasudil, Thiazovivin, or Blebbistatin. In some embodiments, the iblastoid media 1 may alternatively or additionally further comprise a serum replacement component (for example in addition to, or instead of, N2B27) selected from one or more of an insulin-transferrin-selenium component, optionally insulin-transferrin-selenium-ethanolamine (ITS-X) or insulin-transferrin-selenium (ITS-G); serum replacement (SR; Sigma); or knock-out serum replacement (KOSR; Gibco). In some embodiments, the iblastoid media 1 may further comprise one or more of a LIF component, optionally human LIF, bovine LIF, caprine LIF, porcine LIF, buffalo LIF, or recombinant LIF, optionally at a concentration of about 10 ng/ml; an epidermal growth factor (EGF) component, optionally EGF; a GSK33 inhibitor, optionally CHIR99021, BIO, CHIR-98014, LY2090314, or IM-12; a Wnt activator, optionally Wnt3a, Wnt agonist 1, or SKL2001; a histone deacetylase inhibitor (HDAC inhibitor), optionally valproic acid (VPA), sodium butyrate, or trichostatin A, optionally 0.2-0.8 mM VPA; and/or a second TGFβ-1 inhibitor, optionally SB431542, A83-01, LY2109761, SB525334, SB505124, LDN-193189, RepSox, BIBF-0775, TGFβRI-IN-3, or TP0427736. Optionally, the iblastoid media 1 may comprise one or more of N2B27, PD0325901, A83-01, LPA, human LIF, and/or Y27632. In embodiments where the first iblastoid media comprises an EGF component, the first iblastoid media may further comprise an fibroblast growth factor (FGF) component, optionally basic fibroblast growth factor (bFGF) or FGF4, optionally at a concentration of 10-30 ng/ml.

In another embodiment, a second iblastoid media comprises a base medium and/or supplements as well as one or more second iblastoid media components. In an embodiment, the second iblastoid media (also referred to herein as iblastoid media 2) comprises one or more of a serum replacement component, optionally an N2B27 component comprising B27 supplement and N2 supplement, optionally comprising about 0.5% to about 2% B27 supplement, optionally about 1% B27 supplement, and about 0.5% to about 1% N2 supplement, optionally about 0.5% N2 supplement; a TGFβ-1 inhibitor, optionally A83-01, SB431542, LY2109761, SB525334, SB505124, LDN-193189, RepSox, BIBF-0775, TGFβRI-IN-3, or TP0427736; a HIPPO pathway inhibitor, optionally LPA; and a ROCK inhibitor component, optionally Y27632, fasudil, Thiazovivin, or Blebbistatin. In some embodiments, the iblastoid media 2 may alternatively or additionally further comprise a serum replacement component (for example in addition to, or instead of, N2B27) selected from one or more of an insulin-transferrin-selenium component, optionally insulin-transferrin-selenium-ethanolamine (ITS-X) or insulin-transferrin-selenium (ITS-G); serum replacement (SR; Sigma), or knock-out serum replacement (KOSR; Gibco). In some embodiments, the iblastoid media 2 may further comprise one or more of an fibroblast growth factor (FGF) component, optionally basic fibroblast growth factor (bFGF) or FGF4; an Activin A component, optionally human Activin A, bovine Activin A, or murine Activin A; a Wnt activator, optionally Wnt3a, Wnt agonist 1 or SKL2001; and/or a GSK33 inhibitor, optionally CHIR99021, BIO, CHIR-98014, LY2090314, or IM-12. Optionally, the iblastoid medium 2 may comprise one or more of N2B27, A83-01, LPA, and/or Y27632. In embodiments where the second iblastoid media comprises an FGF component, the second iblastoid media may further comprise an epidermal growth factor (EGF) component, optionally about 50 ng/ml EGF.

### III. Use of Iblastoids in Bovine Breeding Programs

The methods for preparing bovine iblastoids described herein can be used to generate a plurality of genetically identical bovine embryos, without cloning, which are useful in bovine breeding programs.

To evaluate the suitability for markets and role within selection and breeding systems, germplasm may be characterized. Embryos, fetuses, outgrowths, stem cell lines, or iblastoids at any stage may be biopsied to remove a few cells (approximately 5 to 10) and the remaining cells may be used fresh or cryopreserved for future use in establishing a cell line and/or iblastoid. Alternatively, a cell line may be established from an entire embryo or iblastoid, then a subset of the cells may be used to perform the genomic and/or epigenetic evaluation. The cell line may be maintained in active culture while waiting for the genomic results or may be frozen and thawed later when one has the genetic and/or predicted performance ability information.

For genomic or epigenetic analysis, DNA is extracted from the biopsied embryonic, fetal, sperm, animal, or iblastoid cells or the established cell line and optionally amplified to obtain sufficient DNA for genomic analysis. When the quantity of DNA collected is insufficient, DNA may be amplified using a DNA amplification protocol adapted for samples that have small quantities of DNA, such as those described by Saadi et al. (2014a) and Saadi et al. (2014b).

In one embodiment of the present disclosure, the target cells (e.g. embryos, outgrowths, stem cells, iblastoids, animals, sperm, oocytes) and/or mating pairs are characterized by testing for one or more biomarkers, and optionally scoring and/or selecting for desired biomarkers (e.g. genetic, genomic, epigenetic, and/or performance ability characteristic(s)).

“Scoring” typically refers to testing for one or more biomarkers for multiple traits or characteristics, then creating a score that is made up of a series of traits that are equally or differentially weighted according to their relative value. Embryos, outgrowths, stem cells, iblastoids, animals, sperm, oocytes, and/or mating pairs may be scored based on the results from testing for one or more biomarkers. A “score” can also refer to a value made up of hundreds or thousands of genomic markers and/or epigenetic marks that span the entire genome and which individually or jointly constitute a genomic prediction of the breeding value, transmitting ability, or performance ability of an individual. In some embodiments, the score is a genomic score, optionally comprising a weighted combination of one or more single nucleotide polymorphisms and/or one or more epigenetic marks. Examples of such scores include, without limitation, genomic breeding values, genomic transmitting ability, or genomic predicted performance ability (gPPA), optionally where the gPPA also may incorporate effects which may impact performance but are not transmitted to future generations (Varona et al., 2018). Optionally, a composite index for scoring may be constructed considering the specific market, environmental, and/or economic conditions, and associated interactions, including those with genotypes (e.g. genotype by environment interactions).

Assessment of homozygosity of the cells or mating pairs and their genomic imputations (Sargolzaei et al., 2014) may be performed initially when obtaining the genomic breeding value from each cell and/or mating pair.

Optionally, other genetic and/or non-genetic inherited effects may be incorporated into genomic selection (Varona et al., 2018; David et al., 2019).

Alternatively, the genomic breeding value may be obtained from the embryonic, iblastoid, fetal, sperm, oocyte, animal, or stem cells, or mating pair without genomic imputation. In other words, with sufficient density of marker genotypes, no markers would need to be deduced from neighboring markers and implied pedigree based on genomic relationships.

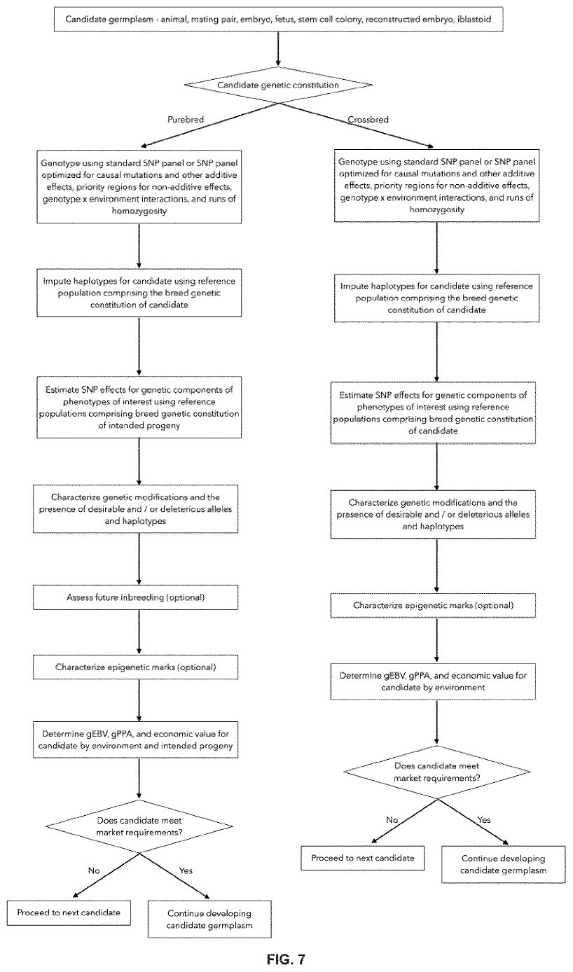

Testing, scoring, and/or selecting also may consider the genetic constitution of target cells (e.g. embryos, outgrowths, stem cells, iblastoids, animals, sperm, oocytes) and/or mating pairs (see FIG. 7). For example, Khansefid et al. (2020) reported, “In conclusion, to improve crossbred GP [genomic prediction], we recommend a balanced-breed reference containing crossbred animals and using a set of SNP [single nucleotide polymorphisms] close to QTL [quantitative trait loci] and enriched for causal mutations.” Accordingly, in one embodiment, testing, scoring, and/or selecting is performed considering one or more specified markets and/or one or more genetic (e.g. breed, familial) constitutions.

For example, candidate sire-dam pairs may be characterized as described above, and the gPPA scores of future offspring may be determined. Optionally, the gPPA score also may incorporate one or more of non-additive genetic effects, homozygosity effects, and effects of epigenetic marks.

Hayes et al. (2009) describe genomic selection as, “Genomic selection refers to selection decisions based on genomic [estimated] breeding values (GEBV). The GEBV are calculated as the sum of the effects of dense genetic markers, or haplotypes of these markers, across the entire genome, thereby potentially capturing all the quantitative trait loci (QTL) that contribute to variation in a trait.” Accordingly, “selection” refers to identifying a subpopulation of embryos, stem cells, iblastoids, animals and/or mating pairs in the population with the most desirable scores and choosing one or more individuals of the identified subpopulation for use in the method and/or breeding program. A subpopulation of animals or mating pairs may be selected on the basis of one or more scores, for example as described above. In an embodiment, selecting is performed considering one or more specified markets and/or one or more genetic (e.g. breed, familial) constitutions. FIG. 7 shows exemplary steps of characterizing (testing, screening, scoring, and selecting) candidate germplasm for its suitability for target markets.

In one embodiment, the cells are scored wherein characterizing the genome and/or epigenetic marks of the cells comprises performing genomic scoring of the cell.

In one embodiment, the mating pairs are scored wherein characterizing the genome and/or epigenetic marks of the mating pairs comprises performing genomic scoring of the mating pair.

In another version of the method, the selection of the cells is after assessment of homozygosity of the mating pairs or cells and/or their genomic imputation and/or estimation of genomic breeding value and/or genomic predicted performance ability of each embryo, fetus, iblastoid, sperm, oocyte, animal, stem cell line, or mating pair.

In one version of the method, testing, scoring, and/or selection is done to:
a) create a subpopulation of iblastoids, naïve stem cells, haploid embryos, animals, etc. enriched for a particular trait, or
b) create a subpopulation of iblastoids, naïve stem cells, haploid embryos, animals, etc. without a particular trait, or
c) create a subpopulation of iblastoids, naïve stem cells, haploid embryos, animals, etc. with a series of favorable traits and/or without a series of unfavorable traits, or
d) create a subpopulation of iblastoids, naïve stem cells, haploid embryos, animals, etc. without one or more deleterious haplotypes, or
e) create breeding animals or breeding lines (iblastoids, naïve stem cells, haploid embryos, etc.) that contain a unique combination of alleles, haplotypes or traits, wherein the alleles, haplotypes or traits are normally appearing at a “low” frequency, typically below 50% of the population but present at least 0.1% or at least 0.01%
f) create a subpopulation of iblastoids, naïve stem cells, haploid embryos, animals, etc. particularly well suited to a specific environment or for a particular market.

In another embodiment of the disclosure, the genetic, genomic, or epigenetic characteristics include one or more production traits (e.g. milk, fat, protein, fat %, protein %, milk protein variant composition e.g. A2A2 milk), meat quality traits, growth traits, health traits (e.g. somatic cell score, mastitis resistance, immune response, livability, disease resistance), reproductive traits (e.g. pregnancy rate, conception rate), calving traits (e.g. calving ease, calving to first insemination, stillbirths), conformation traits (e.g. polled traits, udder and teat traits, feet and leg traits, body traits, dimension traits), efficiency traits (e.g. feed efficiency related, methane efficiency, workability, longevity, productive life), novel traits (e.g. robotic milking traits, heat tolerance, slick haircoat, activity traits and behavior traits), composite index traits (e.g. Life Production Index (LPI), Total Production Index (TPI), Lifetime Net Merit Dollars (NM$)), heterozygosity, and/or the absence of various deleterious alleles and haplotypes (e.g. haplotypes affecting fertility, dwarfism, mulefoot, hypotrichosis, brachyspina, citrullinemia, bovine leukocyte adhesion deficiency).

Once one or more mating pairs are selected based on one or more scores or traits (for example, those described above) they can be used to generate one or more embryos. For example, using standard methods known in the art, oocytes and sperm are collected from females and males, respectively selected with gPPA scores of offspring in the highest/most desirable 50%.

Embryos are produced via in vitro fertilization using collected germ cells from selected mating pairs with the most desirable gPPA scores for offspring. Optionally, embryos may be characterized to determine the gPPA scores (and/or other desired characteristics) of individual embryos.

An embryo (or naïve stem cells derived therefrom) may be selected based on one or more desired characteristics, scores, etc. and then used to generate a plurality of genetically identical iblastoids using the methods described herein. These may then be transferred to recipient female surrogates and gestated to produce genetically identical offspring. The performance of the offspring may then be determined for one or more traits (e.g. milk production traits, meat quality traits, etc. as desired, optionally based on suitability for a given environment or market). Alternatively, or additionally, the phenotypic data from the initial cohorts would be incorporated into analyses in order to compute updated scores. The offspring may then be included in future breeding programs, if desired. Alternatively, or additionally, the iblastoids may be selected and used in an in vitro breeding scheme.

Additional genetically identical iblastoids may also be generated from stored stocks of naïve stem cells and/or iblastoids if desired, for example if the performance and/or updated score of the genetically identical offspring is particularly desirable.

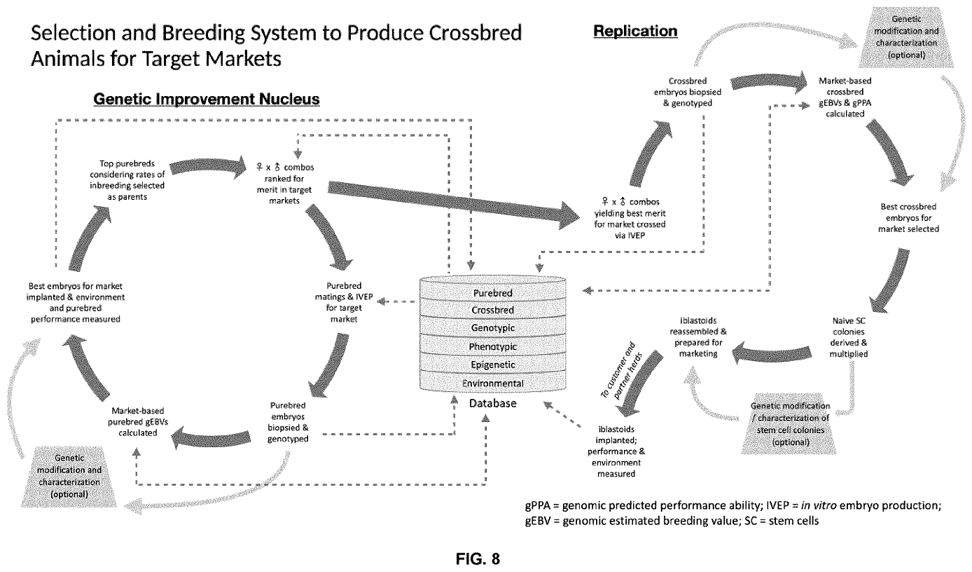

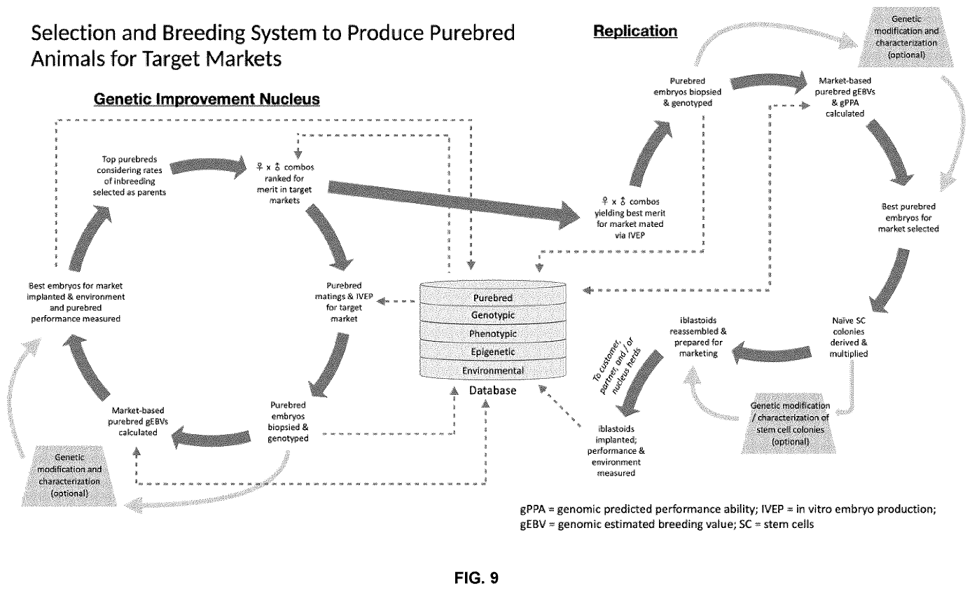

Representative selection and breeding systems with the potential to use the disclosed iblastoid technology for the production of market-specific genetic products including embryos and progeny are outlined in FIG. 8 for crossbreds and FIG. 9 for purebreds. In addition to utilizing molecular and advanced reproductive technologies such as genomic selection, genomic predicted performance ability, oocyte pick up, in vitro embryo production, reassembly of blastocyst-like iblastoids, among others, the systems include optional genetic modification steps. Breeding systems described by others (e.g. Hou et al., 2018; Bishop and Van Eenennam, 2020; Mueller and Van Eenennaam, 2022 to name a few) also may benefit from using the disclosed iblastoid technology when the production of multiple genetically identical embryos is desired.

### IV. Products and Compositions of Matter, and Uses Thereof

In one aspect of the disclosure there are provided products and compositions of matter useful for the aggregation, formation, and/or maintenance of bovine iblastoids.

Also provided are formulations of media suitable for promoting bovine naïve stem cell aggregation, bovine iblastoid formation and/or for preparing bovine iblastoids as described herein.

In one embodiment, the aggregation medium comprises a base medium and one or more components identified herein. For example, in one embodiment, the aggregation medium comprises one or more of a serum replacement component, such as an N2B27 component, and a Rho-associated, coiled-coil containing protein kinase (ROCK) inhibitor component.

In an embodiment, the serum replacement component comprises one or more of a) an N2B27 component; b) an insulin-transferrin-selenium component, optionally insulin-transferrin-selenium-ethanolamine (ITS-X) or insulin-transferrin-selenium (ITS-G); c) serum replacement (SR; Sigma); or d) knock-out serum replacement (KOSR; Gibco). In one embodiment, the N2B27 component comprises B27 supplement and N2 supplement, optionally about 0.5% to about 2% B27 supplement, optionally about 1% B27 supplement, and about 0.5% to about 1% N2 supplement, optionally about 0.5% N2 supplement.

In one embodiment, the ROCK inhibitor component comprises Y27632, fasudil, Thiazovivin, or Blebbistatin. Optionally, the ROCK inhibitor component comprises Y27632, optionally at a concentration of about 0.5 μM to about 20 μM, optionally about 5 μM to about 10 μM, optionally about 5 μM or about 10 μM.

In one embodiment, the iblastoid medium 1 comprises a base medium and one or more components identified herein. For example, in one embodiment, the iblastoid medium 1 comprises a serum replacement component; a MEK inhibitor; a TGFβ-1 inhibitor; a HIPPO pathway inhibitor; and a ROCK inhibitor.

In an embodiment, the serum replacement component comprises a) an N2B27 component; b) an insulin-transferrin-selenium component, optionally insulin-transferrin-selenium-ethanolamine (ITS-X), optionally 0.1-1% ITS-X, or insulin-transferrin-selenium (ITS-G); c) serum replacement (SR; Sigma); or d) knock-out serum replacement (KOSR; Gibco). In one embodiment, the N2B27 component comprises B27 supplement and N2 supplement, optionally about 0.5% to about 2% B27 supplement, optionally about 1% B27 supplement, and about 0.5% to about 1% N2 supplement, optionally about 0.5% N2 supplement.

In one embodiment, the MEK/ERK inhibitor component comprises PD0325901, Ravoxertinib, GSK1120212, MEK162, PD184352, Trametinib, LY3214996, or Ulixertinib. Optionally, the MEK/ERK inhibitor component comprises PD0325901 or Ravoxertinib. Optionally, the MEK/ERK inhibitor component comprises PD0325901 at a concentration of about 0.05 μM to about 5 μM, optionally about 0.1 μM to about 2 μM, optionally about 1 μM. Optionally the MEK/ERK inhibitor component comprises Ravoxertinib at a concentration of about 0.25 μM to about 10 μM, optionally about 1 μM to about 5 μM, optionally about 2.5 μM.

In one embodiment, the TGFβ-1 inhibitor comprises A83-01, SB431542, LY2109761, SB525334, SB505124, LDN-193189, RepSox, BIBF-0775, TGFβRI-IN-3, or TP0427736. Optionally the TGFβ-1 inhibitor component comprises A83-01 at a concentration of about 0.1 μM to about 5 μM, optionally about 0.5 μM to about 2 μM, optionally about 1 μM.

In an embodiment, the HIPPO pathway inhibitor comprises lysophosphatidic acid (LPA), optionally at a concentration of about 0.1 to about 5 μM, optionally about 0.5 μM to 2 μM, optionally about 1 μM.

In one embodiment, the ROCK inhibitor component comprises Y27632, fasudil, Thiazovivin, or Blebbistatin. Optionally, the ROCK inhibitor component comprises Y27632, optionally at a concentration of about 0.5 μM to about 20 μM, optionally about 5 μM to about 10 μM, optionally about 5 μM or about 10 μM.

In one embodiment, the iblastoid media 1 further comprises one or more of a second serum replacement component, optionally ITS-X, optionally 0.1-1% ITS-X; a LIF component, optionally human LIF, bovine LIF, caprine LIF, porcine LIF, buffalo LIF, or recombinant LIF, optionally at a concentration of about 10 ng/ml; an epidermal growth factor (EGF) component, optionally about 50 ng/ml EGF; a Wnt activator, optionally 10-100 ng/mL Wnt3a; a GSK33 inhibitor, optionally 1-2 μM CHIR99021; a histone deacetylase inhibitor (HDAC inhibitor), optionally valproic acid (VPA), sodium butyrate, or trichostatin A, optionally 0.2-0.8 mM VPA; and/or a second TGFβ-1 inhibitor, optionally 0.1-1 μM SB431542, optionally A83-01, LY2109761, SB525334, SB505124, LDN-193189, RepSox, BIBF-0775, TGFβRI-IN-3, or TP0427736. In embodiments where the first iblastoid media comprises an EGF component, the first iblastoid media may further comprise an fibroblast growth factor (FGF) component, optionally basic fibroblast growth factor (bFGF) or FGF4, optionally at a concentration of 10-30 ng/ml.

In one embodiment, the LIF component comprises human LIF, bovine LIF, porcine LIF, caprine LIF, buffalo LIF, or recombinant LIF, optionally at a concentration of about 1 ng/ml to about 1000 ng/ml, optionally about 5 ng/ml to about 100 ng/ml, optionally about 5 ng/ml, about 10 ng/ml, about 20 ng/ml, or about 100 ng/ml.

In one embodiment, the iblastoid medium 2 comprises a base medium and one or more components identified herein. For example, in one embodiment, the iblastoid medium 2 comprises one or more of a serum replacement component, such as an N2B27 component; a TGFβ-1 inhibitor component; a HIPPO pathway inhibitor; and a ROCK inhibitor.

In an embodiment, the serum replacement component comprises one or more of a) an N2B27 component; b) an insulin-transferrin-selenium component, optionally insulin-transferrin-selenium-ethanolamine (ITS-X) or insulin-transferrin-selenium (ITS-G); c) serum replacement (SR; Sigma); or d) knock-out serum replacement (KOSR; Gibco). In one embodiment, the N2B27 component comprises B27 supplement and N2 supplement, optionally about 0.5% to about 2% B27 supplement, optionally about 1% B27 supplement, and about 0.5% to about 1% N2 supplement, optionally about 0.5% N2 supplement.

In one embodiment, the TGFβ-1 inhibitor comprises A83-01, SB431542, LY2109761, SB525334, SB505124, LDN-193189, RepSox, BIBF-0775, TGFβRI-IN-3, or TP0427736. In an embodiment, the TGFβ-1 inhibitor comprises A83-01, optionally at a concentration of about 0.5 μM to about 2 μM, optionally about 1 μM.

In an embodiment, the HIPPO pathway inhibitor comprises lysophosphatidic acid (LPA), optionally at a concentration of about 0.5 μM to about 2 μM, optionally about 1 μM.

In one embodiment, the ROCK inhibitor component comprises Y27632, fasudil, Thiazovivin, or Blebbistatin. Optionally, the ROCK inhibitor component comprises Y27632, optionally at a concentration of about 0.5 μM to about 20 μM, optionally about 5 μM to about 10 μM, optionally about 5 μM or about 10 μM.

In one embodiment, the iblastoid media 2 further comprises one or more of a second serum replacement component, optionally ITS-X, optionally 0.1-1% ITS-X; an fibroblast growth factor (FGF) component, optionally basic fibroblast growth factor (bFGF) or FGF4, optionally at a concentration of 10-30 ng/ml; an Activin A component, optionally 5-50 ng/ml Activin A; a Wnt activator, optionally Wnt3a, Wnt agonist 1, or SKL2001; and/or a GSK3β inhibitor, optionally 1-3 μM CHIR99021. In embodiments where the iblastoid media 2 comprises an FGF component, the iblastoid media 2 may further comprise an epidermal growth factor (EGF) component, optionally about 50 ng/ml EGF.

In one embodiment, the Activin A component comprises human Activin A, bovine Activin A, or murine Activin A, optionally at a concentration of about 1 ng/ml to about 50 ng/ml, optionally about 5 ng/ml to about 50 ng/ml, optionally about 10 ng/ml or about 20 ng/ml.

In one embodiment, the GSK33 inhibitor component comprises CHIR99021, BIO, CHIR-98014, LY2090314, or IM-12. Optionally, the GSK33 inhibitor component comprises CHIR99021, optionally at a concentration of about 0.1 μM to about 5 μM, optionally about 1 μM, to about 3 μM, optionally about 1 μM, about 2 μM, or about 3 μM.

In an embodiment, the Wnt activator component comprises Wnt3a, Wnt agonist 1, or SKL2001. In an embodiment, the Wnt activator component comprises Wnt3a, optionally at a concentration of about 10 ng/mL to about 100 ng/mL.

Also provided are kits comprising one or more of an aggregation media, an iblastoid media 1, and/or an iblastoid media 2 as described herein. In one embodiment, the aggregation media, iblastoid media 1, and/or iblastoid media 2 are packaged in separate containers. In one embodiment, the kit further comprises a microwell device for preparing iblastoids.

Also provided is the use of the products, compositions or kits described herein for promoting the aggregation of naïve stem cells in culture, and/or for the formation of iblastoid structures including bovine iblastoids.

For example, in one embodiment there is provided use of an aggregation medium as described herein for promoting the aggregation of dissociated naïve stem cells, optionally bovine naïve stem cells.

In another embodiment, there is provided use of iblastoid media as described herein, for example iblastoid medium 1 and iblastoid medium 2, for culturing aggregated naïve stem cells and promoting the formation of iblastoids. In one embodiment, the iblastoid media are useful for preparing iblastoids, optionally bovine iblastoids.

The following non-limiting examples are illustrative of the present application:

### EXAMPLES

### Example 1. Preparation of 3D Culture System

Naïve stem cells, when attached on feeder cells or protein matrix proliferate as flat/dome shaped colonies. Generation of 3D structures from attached colonies is not efficient because cells tend to proliferate through the given support structure (scaffold). To generate blastocyst-like structures from attached cells with high efficiency, various 3D culture systems could be used such as hanging drop culture, low attachment cell culture dish, or micro-well device made of synthetic polymers. The first goal is to develop a protocol for generating bovine iblastoids from established bovine naïve stem cells and established genetically modified bovine naïve stem cells. The next step is to adapt the protocol to be suitable for the large-scale production of bovine iblastoids for commercial purposes.

In the Examples following, three different 3D culture systems were tested using bovine trophoblast stem cells (TSCs). One of the reasons for using TSCs for these tests is the ease of culturing TSCs. Trophoblast stem cells when in the right conditions will form the blastocoel cavity, thus TSCs can represent an ideal model for testing techniques for iblastoid production. TSCs can also be obtained from embryos of various stages and qualities, thus providing for a robust testing system.

To avoid any confusion, the protocols described below are all based on the tests performed with TSCs. Then, the protocol for iblastoid generation is described in Example 5.

### Cell Dissociation Protocol (for Providing Dissociated Cells for Use in Examples 2-5)

1. Aspirate culture media from culture dish and wash cells with DPBS without Ca²⁺/Mg²⁺, 3 times.
   Note: Other types of buffer/media, which don't contain Ca²⁺ and Mg²⁺ can be used here such as Ca²⁺/Mg²⁺ free Hank's balanced salt solution (HBSS) or Earl's balanced salt solution (EBSS).
2. Add TrypLE into washed cells and incubate cells for 5 min in a humidified incubator with 5% CO₂, 38.5° C.
   Note: other types of dissociation buffer can be used here such as Accutase, Collagenase IV, and 0.05% trypsin.
3. Do gentle pipetting to dissociate cells into small cell clusters. Transfer them into conical tubes. Add 10 ml of DPBS with Ca²⁺/Mg²⁺. Centrifuge at 300×g for 5 min.
   Note: Other types of buffer/media which contain Ca2+ and Mg2+ can be used here such as Hank's balanced salt solution (HBSS), Earl's balanced salt solution (EBSS), DMEM, DMEM/F12, Advanced DMEM, Advanced DMEM/F12, Neurobasal medium, RPM11640, α-MEM, Opti-MEM, and other cell culture media.
4. Discard supernatant and resuspend cell pellet with 1 ml of fresh N2B27 media.
   Note 1: N2B27 media is a 1:1 mixture of base media DMEM/F12 and Neurobasal medium, supplemented with 1% B27 supplement, 0.5% N2 supplement, 1× Glutamax, 1×NEAA, 0.1 mM β-mercaptoethanol, and 50 IU/ml Pen/Strep.
   Note 2: N2B27 base media can be replaced by DMEM/F12 or Advanced DMEM/F12, and other supplements can be used.
5. Centrifuge at 300×g for 5 min.
6. Discard supernatant and resuspend cell pellet with 1 ml of fresh N2B27 media.

### Example 2. Hanging Drop System

The Hanging drop system is classically used for generating 3D structures from many different types of cells such as mouse embryonic stem cells (Wang and Yang, 2008), Kidney cells (Wang et al., 2017), cancer cells (Foty 2011) and hepatocytes (Shri et al., 2017). To use this approach, small drops of dissociated cells (between 20-30 μl) comprising 100-500 cells are made on the lid of a culture dish. Then, the lid is very carefully flipped back onto the dish to create a hanging drop. This confined environment creates micro gravity inside the drop causing contained cells to settle to the bottom of each drop. After several hours to days, depending on the type of cells (24-48 h), cell aggregations begin forming through confluent contact conditions induced by micro gravity.

Here, a protocol for generating blastocyst-like structures using a hanging drop system is described. Cell lines for all protocols can include genetically selected or genetically selected+modified.

### Hanging Drop Preparation

For the hanging drop method, dissociated cells were used at a concentration of 1×10⁴ cells/mi.
1. Put 20 μl of dissociated cell suspension (200 cells/drop) on the lid of a dish.
2. Add sterilized water to cover the bottom of the dish.
   Note: Any sterile liquid can be used to create a saturated humidity environment, such as normal saline, DPBS, HBSS, EBSS or Milli-Q (ultra-pure) water.
3. Very carefully flip the lid (avoid breaking the adhered drops, see FIG. 1B) and place on top of the dish containing sterilized water.
4. Culture cells in the hanging drop in a humidified incubator with 5% CO₂, 38.5° C. for up to 48 hrs until blastocyst-like structures become visible.
5. After 48 hr, blastocyst-like structures are transferred into regular embryo culture dishes.

### Results

Aggregation efficiency. Each 20 μL hanging drop contains approximately 10-15 aggregated structures.

The size of the structures produced using this method ranged between 50 and 150 μm in diameter.

The target diameter of the blastocyst-like structures is approximately 100-150 μm (similar to the size of a day-7 blastocyst). Using this method, only 1 or 2 structures per drop of 20 μL were in the target range of size.

Another important factor when producing blastocyst-like structures is the presence of the blastocoel cavity. Using the hanging drop method, approximately 50% of structures displayed a cavity.

Taken altogether, the hanging drop method has a relatively low aggregation efficiency (0.03 structure/μL), offers very little control over the sizes of the structures produced, and only a small portion of the structures display a blastocoel cavity.

Summary: Number of blastocyst-like structures produced: 4 dishes (10 drops/dish) can be prepared by one person/1 hr. 2 good structures/drop. Total 80 aggregates/hr (estimated maximum capacity).

Comparative picture of a vesicle stained with Hoechst produced using this method side-by-side with a day-7 embryo is shown in FIGS. 1D and 1E

As seen in FIGS. 1D and 1E, following DNA staining, the structures produced show a cell organization pattern that closely matches what can be observed from a day-7 blastocyst (FIG. 1D: TS organoid—blastocyst-like structure, the absence of the ICM is expected here; 1E: day-7 blastocyst with an ICM that can be easily observed by the high abundance of cells, dotted circle)

### Other Observations

It is noted that when the lid of the dish is flipped back to create the “hanging drop”, drops can be easily disturbed or broken, which makes them unusable. Extensive training is required to obtain reliable results from the hanging drop method. In addition, the hanging-drop method can be labour-intensive possibly rendering it ill-suited for the large-scale production of iblastoids.

Lastly, cells are cultured with 20 μl of media for 48 hours in the hanging-drop method. Without being bound by theory, knowing naïve stem cells have a very active metabolism, the culture conditions are very limiting in terms of nutrients available in the media, which may explain the low yield of blastocyst-like structures.

### Example 3. Low Attachment Dish Method

A low attachment dish method can be used as an alternative method to the hanging drop system. It was successfully used with many different types of cells such as embryonic stem cell differentiation (Valamehr et al., 2008), liver organoids (Thompson and Takebe, 2020), and cerebral organoids (Giandomenico et al., 2021). When coated with a hydrogel layer, the surface of low attachment dishes completely inhibits cellular attachment. Because cells cannot attach to the coated dish, they will grow as 3D structures in a suspension culture system. A low attachment dish method has many potential advantages compared to a hanging drop method, in particular because there is no need to prepare individual drops, processing speed is greatly increased.

### Protocol for Low Attachment Dish Method

For the low attachment dish method, dissociated cells were used at concentration of 1×10⁶ cells/ml.
1. Add 500 μl of fresh N2B27 media into each well of non-adherent dish, such as Corning, Cat #3473; NUNC, Cat #174930; Sciencell, Cat #0383.
2. Put 100 μl of dissociated cells (1×10⁵ cells/well) into each well.
3. Culture cells in a humidified incubator with 5% CO₂, 38.5° C. for 48 hours until blastocyst-like structures become visible.
4. After 48 hr of culture, blastocyst-like structures are transferred into embryo culture dishes.

### Results

Aggregation efficiency: Each well contains approximately 300 good structures/500 μL (volume of culture media contained in 1 well).

The size of structures produced using this method ranged from 50 to 150 μm in diameter with 80% of structures having a diameter of approximately 100 to 150 μm.

When looking at the proportion of structures having a blastocoel cavity, 80% of structures displayed a cavity.

Taken altogether, the low attachment dish method shows greater aggregation efficiency (0.38 structure/μL) compared to the hanging drop method (0.03 structure/μL), and also a higher efficiency of cavity formation (80% vs. 50%). However, a large degree of variability in the size of the structures is observed.

To produce structures with a more homogenous size and more importantly a size that closely matches that of day-7 embryos, a different technique may be required.

### Example 4. Microwell System

While the methods of Examples 2 and 3 are frequently used to produce relatively large organoids (scale of mm), researchers have developed other methods to create much smaller structures (range of μm). Micro-well systems have been used to generate cell aggregations especially for embryonic body formation (Antonchuk 2013) or adult stem cell spheroid generation (Futrega et al., 2017). Recently, a group working with human embryonic stem cells has used a micro-well system to generate human iBlastoids (Yu et al., 2021, Liu et al., 2021). It was hypothesized that wells of defined size will provide consistent culture conditions for cells, and as a result will yield aggregates of homogenous size. Therefore, microwell systems were tested for their ability to improve the size homogeneity of the resulting bovine blastocyst-like structures.

### Protocol

1. Prepare microwell device by adding 1 ml of 0.1% BSA in N2B27 media into a StemFIT 3D™ microwell device (Microfit, Cat #H853400) followed by incubating overnight in a humidified incubator with 5% CO₂, 38.5° C. (FIG. 3)
2. Prepare cells using the Cell dissociation protocol described in Example 1.
3. Dilute cell pellet to obtain a concentration of 1×10⁵/ml.
4. Add dissociated cells into the microwell device to obtain approximately 20-60 cells/well.
5. Cell aggregates can be observed after 24 hrs of culture in a humidified incubator with 5% CO₂, 38.5° C. and structures with blastocoel cavity are visible after 48 hrs of culture.

### Results

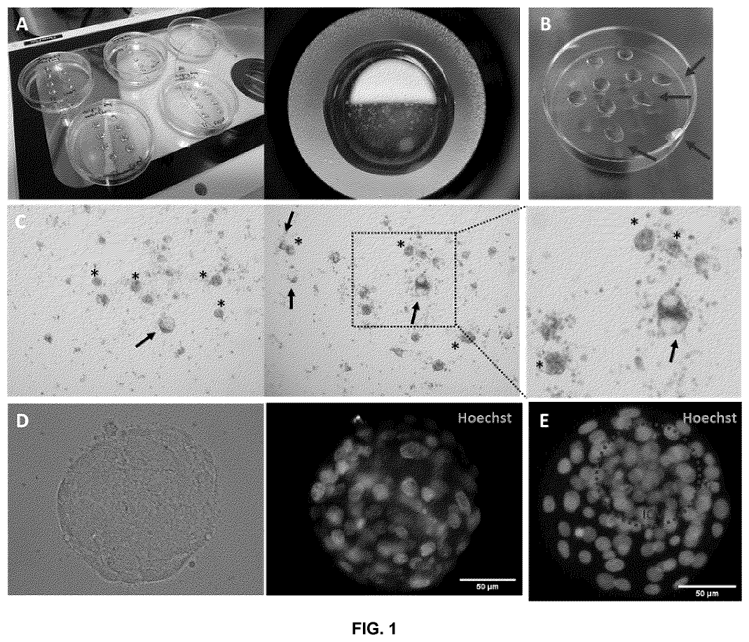

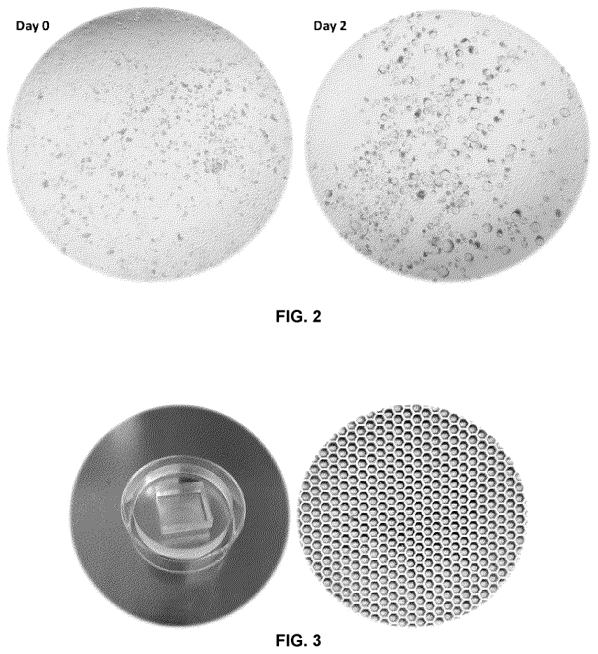

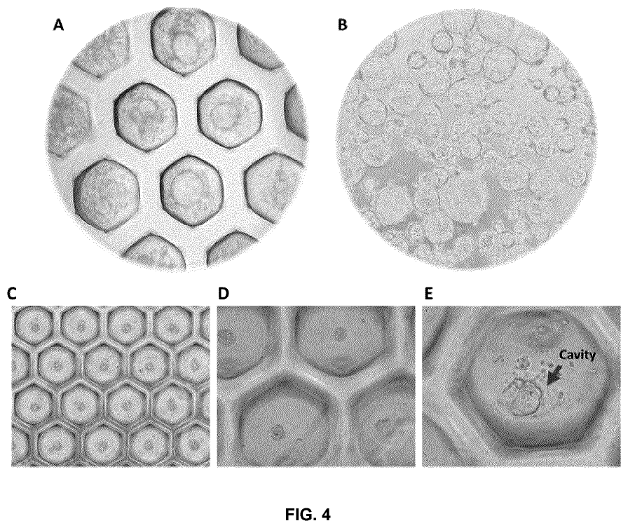

### Aggregation Efficiency (FIG. 4)

Each device contains approximately 600 good structures/1 mL (volume of device).

The size of the structures produced using this method ranged between 50 and 150 μm in diameter, where 70% of structures had a diameter of approximately 100 to 150 μm.

A blastocoel cavity was observed in approximately 75% of the structures produced.

Taken altogether, the microwell system demonstrates a similar aggregation efficiency (0.32 structure/μl) compared to the low attachment culture method (0.38 structure/μl), and less variability in the size of the structures produced.

Without wishing to be bound by theory, because the initial number of cells and size of the culture space is similar for all wells, the resulting blastocyst-like structures show a greater degree of homogeneity in size than when using a low attachment dish approach.

A microwell system requires a similar production time and yields a larger number of homogenously sized blastocyst-like structures when compared to a low attachment dish approach. Another advantage may be related to the abundance of nutriments for cells when the volume of the microwell is approximately 5 μL and all wells “share” a total of 1 mL of culture media (vs. 20 μl; hanging drop or 500 μl; low attachment methods).

### Example 5. Generation of Iblastoids

Naïve embryonic stem cells may originate from preimplantation ICM cells or early morula blastomeres, which can then differentiate into TE cells (Dong et al., 2020) and extraembryonic endoderm cells (XEN, Linneberg-Agerholm et al., 2019). In 2021, two studies reported the generation of human iblastoids (Liu et al., 2021 and Yu et al., 2021) by applying TE and XEN differentiation techniques to naïve pluripotent stem cells. Briefly, without wishing to be bound by theory, naïve stem cells may be induced into TE lineage by inhibiting the MEK/ERK and TGFβ-1 pathways. Inhibition of the HIPPO pathway may induce the polarization of TE cells by activation of TEAD4 transcription factor and induce cavity formation into iblastoids. Then, the activation of the MEK/ERK pathway through the removal of MEK inhibitor may induce the priming of naïve stem cells followed by endodermal differentiation.

The protocol was as follows:
1. (Day 0) Bovine naïve stem cells were produced as described in Patent Cooperation Treaty Application No. PCT/CA2022/051664. Cells were dissociated as described in Example 1 and resuspended in N2B27 media with 5-10 μM Y27632. Note: iPSC-derived naïve stem cells, primed embryonic stem cell derived naïve stem cells, embryo derived expanded naïve stem cells, or other suitably derived naïve stem cells can also be used, and/or cells may be genetically modified/genome edited.
2. 20-30 individual naïve stem cells were added to each well of a StemFIT 3D™ microwell device (Microfit, Cat #H853400) with N2B27 media+5-10 μM Y27632 (ROCK inhibitor) and cultured in a humidified incubator with 6.8% CO₂, 5% O₂ at 38.5° C. for 24 hours.
   Note: ROCK inhibitor may reduce cell death induced by mechanical damages (Chen 2010).
3. After 24 hours, media and non-aggregated cells were removed from the microwell device by gentle pipette aspiration.
4. iblastoid media 1 (N2B27 basal media+1 μM PD0325901 (MEK inhibitor)+1 μM A83-01 (TGFβ-1 inhibitor)+1 μM LPA (lysophosphatidic acid, HIPPO pathway inhibitor)+10 ng/ml LIF (leukemia inhibitory factor)+5-10 μM Y27632 (ROCK inhibitor)) were added. Optional additives may improve the efficiency: 0.1-1% ITS-X (insulin-transferrin-selenium-ethanolamine), 50 ng/ml EGF (epidermal growth factor), 1-2 μM CHIR99021 (GSK33 inhibitor), 0.2-0.8 mM VPA (a histone deacetylase inhibitor, valproic acid), or 0.1-1 μM SB431542 (TGFβ-1 inhibitor).
5. Microwell devices were incubated in a humidified incubator with 6.8% CO₂, 5% O₂ at 38.5° C. for up to 24 hrs.
6. Media change with iblastoid media 1 was repeated on Day 2.
7. On Day 3 (after about 48 hours in iblastoid media 1), media was changed to iblastoid media 2 (N2B27 basal media+1 μM A83-01+1 μM LPA+5-10 μM Y27632). Optional additives may improve the efficiency: a second serum replacement component, such as 0.1-1% ITS-X; 10-30 ng/ml bFGF (basic fibroblast growth factor), 5-50 ng/ml Activin A, or 1-3 μM CHIR99021. Samples were incubated in a humidified incubator with 6.8% CO₂, 5% O₂ at 38.5° C. for up to 24 hrs
8. On Day 4, media change with iblastoid media 2 was repeated.

### Evaluation of Iblastoids (S/C/S Staining).

Embryos consist of different types of cells depending on the developmental stage. Day-7 embryos primarily comprise two types of cells (ICM and TE) and day-9 to day-15 embryos comprise three types of cells (ICM, TE, and hypoblast). To detect the presence of each cell type in iblastoids, morphological evaluation was performed on day-5 iblastoids and compared to morphology and S/C/S staining of day-7 and day-11 embryos stained according to standard protocols.

The protocol for S/C/S staining of iblastoids was as follows:
1. iblastoids were washed with DPBS with Ca²⁺/Mg²⁺, 3 times.
2. iblastoids were fixed with cold 2-4% paraformaldehyde (PFA) for 15 min at room temperature.
3. Samples were washed with DPBS without Ca²⁺/Mg²⁺ for 5 min, 3 times
4. Samples were permeabilized with 0.5% Triton X in DPBS without Ca²⁺/Mg²⁺ for 30 min to 1 hour.
5. Samples were incubated in blocking solution (1-3% BSA in DPBS without Ca²⁺/Mg²⁺) for 1 hour.
6. Samples were incubated at 4° C. overnight with primary antibodies (SOX2 [Invitrogen, 14-9811-82] for ICM; CDX2 [Biogenex, AM382] for TE; and SOX17 [R&D system, AF1924] for hypoblast) in blocking solution.
7. Samples were washed with DPBST (0.05% Twin20 in DPBS without Ca²⁺/Mg²⁺) for 5 min, 3 times.
8. Samples were incubated for 1 hr at room temperature with secondary antibodies in DPBST.
9. Samples were washed with DPBST (0.05% Twin20 in DPBS without Ca²⁺/Mg²⁺) for 5 min, 3 times.
10. Samples were deposited on the slide glass and covered with mounting media (Vectashield+DAPI).

### Results

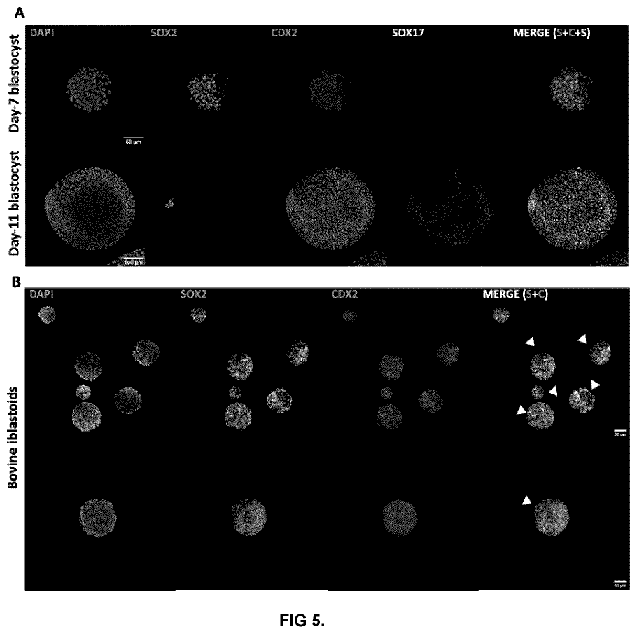

Each type of cell in embryos (ICM, TE, and hypoblast) can be detected by S/C/S staining (FIG. 5A) in day-11 embryos.

SOX2 (ICM marker) and CDX2 (TE marker) are detected from day-7 and day-11 embryos. However, SOX17 (hypoblast marker) is detected beginning from Day 9 (not shown) with very clear signals obtained from day-11 embryos as hypoblast cells begin appearing after expansion of the blastocyst.

Taken together, S/C/S staining shows good visualization of embryos with high specificity for each desired cell type. This tool can be potentially used for evaluation of iblastoids.

The structure of bovine iblastoids can be further recognized by SOX2 and CDX2 staining (FIG. 5B). After 5 days of induction, bovine naïve stem cell aggregates express SOX2 and CDX2. Each aggregate has a cavity-like structure which lacks expression of SOX2 and has fewer cells (arrowhead in FIG. 5B). The size of iblastoids is similar to day-7 embryos (FIG. 5, scale: 50 μm).

### Example 6. Generation of a Plurality of Genetically Identical Iblastoids from an Individual Mating

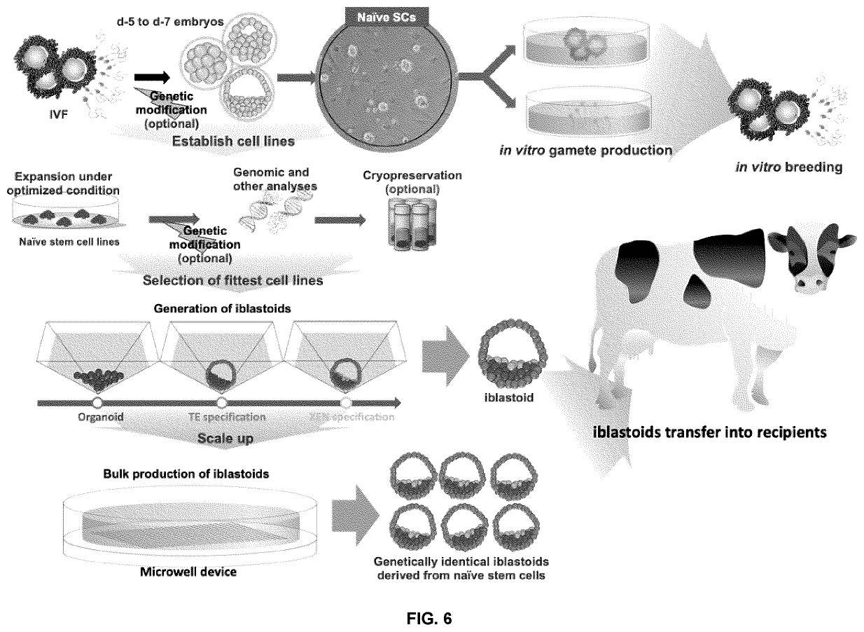

When used in conjunction with genomic selection, other assisted reproductive technologies, and/or in vitro breeding (such as Hou et al., 2018), iblastoid technology reported herein will provide additional opportunities for improving system genetic gain via increased selection intensity, shortened generation intervals, and reduced genetic lag. Thus, as shown in FIGS. 6-9, iblastoid technology has the potential to supplant somatic cell nuclear cloning (SCNT) or AI and provide significant value for breeding systems and commercial settings particularly for animal species with relatively low fecundity and moderate to long generation intervals by providing an efficient platform for producing genetically modified animals at scale, when specific genomes are required, for crossbreeding, when specific breed compositions are desired (e.g. F1, 3-way cross, 5/8:3/8, among others), for replicating important constituents and products within traditional and in vitro breeding systems, and improving the delivery of advanced veterinary medical biologics and therapeutics.

As shown in FIG. 8, the iblastoid generation methods described above are utilized to generate a plurality of genetically identical iblastoids from an individual mating as follows:
1. One or more candidate sire/dam pairs are optionally characterized or assessed for desired traits, for example by testing for one or more biomarkers (e.g. SNP analysis) and optionally further scored and/or selected based on one or more selection criteria (e.g. estimated breeding value, genomic predicted performance ability, etc.)
2. Oocytes and sperm are collected from selected females and males using techniques known in the art
3. One or more embryos are produced via in vitro fertilization using techniques known in the art
4. Individual embryos are optionally characterized or assessed for desired traits, for example by testing for one or more biomarkers, and optionally further scored and/or selected based on one or more selection criteria (e.g. estimated breeding value, genomic predicted performance ability, etc.)
5. Naïve stem cells are derived and multiplied using methods for example as taught in Patent Cooperation Treaty Application No. PCT/CA2022/051664, or methods for expanded pluripotent stem cells as described by Zhao et al. (2021)
6. Naïve stem cells are optionally characterized or assessed for desired traits, for example by testing for one or more biomarkers, and optionally further scored and/or selected based on one or more selection criteria (e.g. estimated breeding value, genomic predicted performance ability, etc.), and/or a subset of naïve stem cells will optionally be frozen for storage
7. A plurality of genetically identical iblastoids are generated from the naïve stem cells using the methods described herein, for example as described in Example 5
8. One or more iblastoids are optionally characterized or assessed for desired traits, for example by testing for one or more biomarkers, and optionally further scored and/or selected based on one or more selection criteria (e.g. estimated breeding value, genomic predicted performance ability, etc.)

The genetically identical iblastoids are then frozen for storage, and/or transferred to recipient female surrogates and gestated to produce genetically identical offspring. Optionally, the performance of the offspring and/or updated scores are then determined (for example for a given environment or market), and additional genetically identical iblastoids are generated from stored stocks of selected naïve stem cells and/or iblastoids.

### Example 7. Generation of Multiple Genetically Identical Embryos and Use in Selection and Breeding Systems

In light of the scope and scale of global livestock segments, many species experience limited fecundity rates in one or both sexes. This means that traditional selection and breeding systems have limited capacity for assessing and subsequently distributing germplasm that is suited to many specified markets. By enabling the generation in vitro of large numbers of identical iblastoids, iblastoid technology will greatly improve the capacity to deliver offspring with desired performance ability when specific genomes (e.g. offspring from a given mating pair, offspring with one or more desirable characteristics such as resistance to a given disease and/or high methane efficiency, genome edited offspring, among others) and/or specific breed compositions are desired (e.g. F1, 3-way cross, 5/8:3/8, among others).

Selected iblastoids are generated as in Example 6.

Genetically identical iblastoids of good quality are either frozen for future transfer, or transferred fresh to the reproductive tracts of recipient females (surrogates) within one or more specified markets to obtain gestations and viable offspring using conventional embryo transfer protocols.

Offspring are optionally tested for one or more biomarkers (for example to confirm initial testing for one or more biomarkers). Alternatively or additionally, performance data (e.g. milk production etc.) of the offspring in the one or more specified markets are recorded and optionally are used to update scores.

### Results

Table 1 illustrates the improvement in the accuracy of predicted performance of a new iblastoid for three different traits when generating and testing increasingly larger numbers of genetically identical cohort iblastoids.

TABLE 1
Accuracy of predicted performance of a new iblastoid
for three different traits from testing various numbers
of genetically identical cohort iblastoids.

| | Trait | | | |
|---|---|---|---|---|
| | Calving | Milk | Cow | Lifetime Weight of |
| | Interval | Yield | Weight | Calves Weaned |
| heritability (hₐ²) | 0.05 | 0.28 | 0.53 | 0.10 |
| dominance (h_{d}²) | 0.0 | 0.05 | 0.03 | 0.2 |
| No. Iblastoids | | | | |
| tested | | | | |
| 1 | 0.05 | 0.33 | 0.56 | 0.30 |
| 2 | 0.07 | 0.40 | 0.63 | 0.37 |
| 5 | 0.10 | 0.48 | 0.70 | 0.45 |
| 10 | 0.13 | 0.52 | 0.72 | 0.49 |
| 15 | 0.15 | 0.54 | 0.73 | 0.51 |
| 20 | 0.16 | 0.55 | 0.73 | 0.52 |
| 30 | 0.17 | 0.56 | 0.74 | 0.53 |

Computational formulas from Van Vleck, 1999.

Table 2 illustrates the accuracy of predicted mean performance of an iblastoid cohort for three different traits when generating and testing increasingly larger numbers of genetically identical cohort iblastoids.

TABLE 2
Accuracy of predicted mean performance of an iblastoid
cohort for three different traits from testing various
numbers of genetically identical cohort iblastoids.

| | Trait | | | |
|---|---|---|---|---|
| | Calving | Milk | Cow | Lifetime Weight of |
| | Interval | Yield | Weight | Calves Weaned |
| heritability (hₐ²) | 0.05 | 0.28 | 0.53 | 0.10 |
| dominance (h_{d}²) | 0.0 | 0.05 | 0.03 | 0.2 |
| No. Iblastoids | | | | |
| tested | | | | |
| 1 | 0.22 | 0.57 | 0.75 | 0.55 |
| 2 | 0.31 | 0.70 | 0.85 | 0.68 |
| 5 | 0.46 | 0.84 | 0.93 | 0.83 |
| 10 | 0.59 | 0.91 | 0.96 | 0.90 |
| 15 | 0.66 | 0.94 | 0.97 | 0.93 |
| 20 | 0.72 | 0.95 | 0.98 | 0.95 |
| 30 | 0.78 | 0.97 | 0.99 | 0.96 |

Computational formulas from Van Vleck, 1999.

### REFERENCES

Antonchuk J (2013) Formation of embryoid bodies from human pluripotent stem cells using AggreWell™ plates. Methods Mol Biol. 946) 523-533
Bogliotti Y S, Wu J, Vilarino M, Okamura D, Soto D A, Zhong C, Sakurai M, Sampaio R V, Suzuki K, Izpisua Belmonte J C et al. (2018) Efficient derivation of stable primed pluripotent embryonic stem cells from bovine blastocysts. Proceedings of the National Academy of Sciences of the United States of America 115 2090-2095.
Bishop T F and Van Eenennaam A L. (2020) Genome editing approaches to augment livestock breeding programs. J Exp Biol. 223 (Suppl_1): jeb207159. doi: 10.1242/jeb.207159.
Chen G, Hou Z, Gulbranson D R, Thomson J A (2010) Actin-Myosin Contractility Is Responsible for the Reduced Viability of Dissociated Human Embryonic Stem Cells. Cell stem cell. 7 240-248
David I, Canario L, Combes S, and Demars J. 2019. Intergenerational Transmission of Characters Through Genetics, Epigenetics, Microbiota, and Learning in Livestock. Front Genet. 10:1058. doi: 10.3389/fgene.2019.01058
Dong C, Beltcheva M, Gontarz P, Zhang B, Popli P, Fischer L A, Khan S A, Park K M, Yoon E J, Xing Xiaoyun et al. (2020) Derivation of trophoblast stem cells from naïve human pluripotent stem cells. Elife. 9 e52504
Foty R (2011) A simple hanging drop cell culture protocol for generation of 3D spheroids. J Vis Exp. 51 2720
Futrega K, Atkinson K, Lott W B, and Doran M R (2017) Spheroid Coculture of Hematopoietic Stem/Progenitor Cells and Monolayer Expanded Mesenchymal Stem/Stromal Cells in Polydimethylsiloxane Microwells Modestly Improves In Vitro Hematopoietic Stem/Progenitor Cell Expansion. Tissue Eng Part C methods. 23(4) 200-218
Giandomenico S L, Sutcliffe M, and Lancaster M A (2021) Generation and long-term culture of advanced cerebral organoids for studying later stages of neural development. Nat Protoc. 16(2) 579-602
Guo, G, von Meyenn, F, Rostovskaya, M, Clarke, J, Dietmann, S, Baker, D, Sahakyan, A, Myers, S, Bertone, P, Reik, W, Plath K, and Smith A. (2017). Epigenetic resetting of human pluripotency. Development 144, 2748-2763. https://doi.org/10.1242/dev.146811
Han X, Han J, Ding F, Cao S, Lim S S, Dai Y, Zhang R, Zhang Y, Lim B, Li N. (2011). Generation of induced pluripotent stem cells from bovine embryonic fibroblast cells. Cell Res. 21:1509-1512. doi: 10.1038/cr.2011.125
Hayes B J, Bowman P J, Chamberlain A J, and Goddard M E. 2009. Genomic selection in dairy cattle: Progress and challenges. J Dairy Sci. 92:433-443. doi: 10.3168/jds.2008-1646
Hill J R (2014) Incidence of abnormal offspring from cloning and other assisted reproductive technologies Annu. Rev. Anim. Biosci. 2 307-321.
Hou Z, An L, Han J, Yuan Y, Chen D, and Tian J. (2018) Revolutionize livestock breeding in the future: an animal embryo-stem cell breeding system in a dish. J Anim Sci and Biotech. 2018 9:1 9 1-11.
Kagawa H, Javali A, Khoei H H, Sommer™, Sestini G, Novatchkova M, Reimer Y S, Castel G, Bruneau A, Maenhoudt N et al. (2022) Human blastoids model blastocyst development and implantation. Nature 601 600-605.
Khansefid M, Goddard M E, Haile-Mariam M, Konstantinov K V, Schrooten C, de Jong G, Jewell E G, O'Connor E, Pryce J E, Daetwyler H D, and MacLeod IM. 2020. Improving Genomic Prediction of Crossbred and Purebred Dairy Cattle. Front Genet. 11:598580. doi: 10.3389/fgene.2020.598580
Linneberg-Agerholm M, Wong Y F, Herrera J A R, Monteiro R S, Anderson K G V and Brickman J M. (2019) Naïve human pluripotent stem cells respond to Wnt, Nodal and LIF signalling to produce expandable naïve extra-embryonic endoderm. Human development 146(24) dev180620.
Liu X, Tan J P, Schröder J, Aberkane A, Ouyang J F, Mohenska M, Lim S M, Sun Y B Y, Chen J, Sun G et al. (2021) Modelling human blastocysts by reprogramming fibroblasts into iBlastoids. Nature 591 627-632.
Mueller M L and Van Eenennaam A L. (2022). Synergistic power of genomic selection, assisted reproductive technologies, and gene editing to drive genetic improvement of cattle. CABI Agri and Biosci. 3:13.
Park F. (2007) Lentiviral vectors: are they the future of animal transgenesis? Physiol Genomics. 31:159-173.
Perez-Gómez A, Gonzalez-Brusi L, Bermejo-Alvarez P, and Ramos-lbeas P. (2021) Lineage differentiation markers as a proxy for embryo viability in farm ungulates. Front. Vet. Sci. 8 680539
Saadi H A S, O'Doherty A M, Gagne D, Fournier E, Grant J R, Sirard M A, and Robert C. (2014a) An integrated platform for bovine DNA methylome analysis suitable for small samples. BMC Genomics. 15:451.
Saadi H A S, Vigneault C, Sargolzaei M, Gagne D, Fournier E, de Montera B, Chesnais J, Blondin P, and Robert C. (2014b) Impact of whole-genome amplification on the reliability of pre-transfer cattle embryo breeding value estimates. BMC Genomics. 15:889
Sargolzaei M, Chesnais J P, and Schenkel F S. 2014. A new approach for efficient genotype imputation using information from relatives. BMC Genomics. 15:478. doi: 10.1186/1471-2164-15-478
Shri M, Agrawal H, Rani P, Singh D and Onteru S K (2017) Hanging drop, a best three-dimensional (3D) culture methos for primary buffalo and sheep hapatocytes. Scientific reports. 7 1203
Soto D A, Navarro M, Zheng C, Halstead M M, Zhou C, Guiltinan C, Wu J and Ross P J (2021). Simplification of culture conditions and feeder-free expansion of bovine embryonic stem cells. Scientific Reports 2021 11:1 11 1-15.
Springer C, Wolf E, and Simmet K (2021) A new toolbox in experimental embryology—Alternative model organisms for studying preimplantation development. J. Dev. Biol. 9 15
Thompson W L, and Takebe T (2020) Generation of multi-cellular human liver organoids from pluripotent stem cells. Methods Cell Biol. 159 47-68
U.S. Pat. No. 7,067,713 B2
Valamehr B, Jonas S J, Polleux J, Qiao R, Guo S, Gschweng E H, Stiles B, Kam K, Luo T, Witte O N et al. (2008) Hydrophobic surfaces for enhanced differentiation of embryonic stem cell-derived embryoid bodies. PNAS 105(38) 14459-14464
Van Vleck L D. 1999. Implications of cloning for breed improvement strategies: are traditional methods of animal improvement obsolete? J Anim Sci. 77(Suppl 2):111.
Varona L, Legarra A, Toro M A, and Vitezica Z G. 2018. Non-additive Effects in Genomic Selection. Front Genet. 9:78. doi: 10.3389/fgene.2018.00078
Wang S, Wang X, Boone J, Wie J, Yip K.-P, Zhang J, Wang L and Liu R (2017) Application of hanging drop technique for kidney tissue culture. Kidney blood press res. 42 220-231.
Wang X and Yang P (2008) In vitro differentiation of mouse embryonic stem cells using the hanging drop method. J Vis Exp. 17 828
WO 2020/168422 A1
Yanagida A, Spindlow D, Nichols J, Dattani A, Smith A and Guo G (2021) Naïve stem cell blastocyst model captures human embryo lineage segregation. Cell Stem Cell 28.
Yu L, Wei Y, Duan J, Schmitz D A, Sakurai M, Wang L, Wang K, Zhao S, Hon G C and Wu J (2021) Blastocyst-like structures generated from human pluripotent stem cells. Nature 591 620-626.
Zhao L, Gao X, Zheng Y, Wang Z, Zhao G, Ren J, Zhang J, Wu J, Wu B, Chen Y, Sun W, Li Y, Su J, Ding Y, Gao Y, Liu M, Bai X, Sun L, Cao G, Tang F, Bao S, Liu P, and Li X. (2021). Establishment of bovine expanded potential stem cells. Proc Natl Acad Sci USA. 118:e2018505118. doi: 10.1073/pnas.2018505118.

1. A method of preparing bovine iblastoids, the method comprising:
a) providing a population of bovine naïve stem cells;
b) culturing the bovine naïve stem cells in aggregation media under aggregation conditions to produce aggregated bovine naïve stem cells;
c) introducing a first iblastoid media, optionally wherein the aggregation media and non-aggregated cells are removed before introducing the first iblastoid media;
d) culturing the aggregated bovine naïve stem cells in the first iblastoid media under hypoxic conditions;
e) introducing a second iblastoid media, optionally wherein the first iblastoid media is removed before introducing the second iblastoid media; and
f) culturing the aggregated bovine naïve stem cells in the second iblastoid media under hypoxic conditions to obtain the bovine iblastoids.

2. The method of claim 1, wherein the population of bovine naïve stem cells in step a) are dissociated bovine naïve stem cells and/or the bovine naïve stem cells are bovine embryo-derived naïve stem cells.

3. The method of claim 1, wherein step b) comprises introducing the cells into one or more wells of a microwell device and culturing the cells for about 24 hours, optionally wherein the wells of the microwell device have a diameter of about 400 μm to about 600 μm.

4. (canceled)

5. The method of claim 3, wherein the population of bovine naïve stem cells is added to the microwell device at a concentration of approximately 20-60 cells/well, optionally at a concentration of approximately 20-30 cells/well.

6. The method of claim 1, wherein the cells are cultured in step d) and/or step f) for about 48 hours.

7. The method of claim 1, wherein the aggregation media comprises:
a serum replacement component; and
a Rho-associated, coiled-coil containing protein kinase (ROCK) inhibitor component.

8. The method of claim 7, wherein the serum replacement component comprises a) an N2B27 component, optionally about 1% B27 component and about 0.5% N2 component; b) an insulin-transferrin-selenium component, optionally insulin-transferrin-selenium-ethanolamine (ITS-X) or insulin-transferrin-selenium (ITS-G); c) serum replacement (SR); or d) knock-out serum replacement (KOSR); and/or the ROCK inhibitor component comprises Y27632, fasudil, Thiazovivin, or Blebbistatin, optionally 5-10 μM Y27632.

9. The method of claim 1, wherein the first iblastoid media comprises:
a serum replacement component;
a MEK/ERK inhibitor component;
a TGFβ-1 inhibitor component;
a HIPPO pathway inhibitor component; and
a ROCK inhibitor component.

10. The method of claim 9, wherein
the serum replacement component comprises a) an N2B27 component, optionally about 1% B27 supplement and about 0.5% N2 supplement; b) an insulin-transferrin-selenium component, optionally insulin-transferrin-selenium-ethanolamine (ITS-X) or insulin-transferrin-selenium (ITS-G); c) serum replacement (SR); or d) knock-out serum replacement (KOSR);
the MEK/ERK inhibitor component comprises PD0325901, Ravoxertinib, GSK1120212, MEK162, PD184352, Trametinib, LY3214996, or Ulixertinib, optionally about 1 μM PD0325901;
the TGFβ-1 inhibitor component comprises A83-01, SB431542, LY2109761, SB525334, SB505124, LDN-193189, RepSox, BIBF-0775, TGFβRI-IN-3, or TP0427736, optionally about 1 μM A83-01;
the HIPPO pathway inhibitor component comprises lysophosphatidic acid (LPA), optionally about 1 μM LPA; and/or
the ROCK inhibitor component comprises Y27632, fasudil, Thiazovivin, or Blebbistatin, optionally 5-10 μM Y27632.

11. The method of claim 9, wherein the first iblastoid media further comprises a second serum replacement component, optionally ITS-X, optionally 0.1-1% ITS-X; a LIF component, optionally human LIF, bovine LIF, caprine LIF, porcine LIF, buffalo LIF, or recombinant LIF, optionally at a concentration of about 10 ng/ml; an epidermal growth factor (EGF) component, optionally about 50 ng/ml EGF; a Wnt agonist, optionally Wnt3a, Wnt agonist 1, or SKL2001, optionally 10-100 ng/mL Wnt3a; a GSK3β inhibitor, optionally CHIR99021, BIO, CHIR-98014, LY2090314, or IM-12, optionally 1-2 μM CHIR99021; a histone deacetylase inhibitor, optionally valproic acid (VPA), sodium butyrate, or trichostatin A, optionally 0.2-0.8 mM VPA; and/or a second TGFβ-1 inhibitor, optionally 0.1-1 μM SB431542.

12. The method of claim 11, wherein the first iblastoid media comprises an EGF component and further comprises an fibroblast growth factor (FGF) component, optionally basic fibroblast growth factor (bFGF) or FGF4, optionally at a concentration of 10-30 ng/ml.

13. The method of claim 1, wherein the second iblastoid media comprises:
a serum replacement component;
a TGFβ-1 inhibitor component;
a HIPPO pathway inhibitor component; and
a ROCK inhibitor component.

14. The method of claim 13, wherein
the serum replacement component comprises a) an N2B27 component, optionally about 1% B27 supplement and about 0.5% N2 supplement; b) an insulin-transferrin-selenium component, optionally insulin-transferrin-selenium-ethanolamine (ITS-X) or insulin-transferrin-selenium (ITS-G); c) serum replacement (SR); or d) knock-out serum replacement (KOSR);
the TGFβ-1 inhibitor component comprises A83-01, SB431542, LY2109761, SB525334, SB505124, LDN-193189, RepSox, BIBF-0775, TGFβRI-IN-3, or TP0427736, optionally about 1 μM A83-01;
the HIPPO pathway inhibitor component comprises lysophosphatidic acid (LPA), optionally about 1 μM LPA; and/or
the ROCK inhibitor component comprises Y27632, fasudil, Thiazovivin, or Blebbistatin, optionally 5-10 μM Y27632.

15. The method of claim 13, wherein the second iblastoid media further comprises one or more of a second serum replacement component, optionally ITS-X, optionally 0.1-1% ITS-X; an fibroblast growth factor (FGF) component, optionally basic fibroblast growth factor (bFGF) or FGF4, optionally at a concentration of 10-30 ng/ml; an Activin A component, optionally human Activin A, bovine Activin A, or murine Activin A, optionally at a concentration of 5-50 ng/ml; and/or a GSK33 inhibitor, optionally CHIR99021, BIO, CHIR-98014, LY2090314, or IM-12, optionally 1-3 μM CHIR99021,
optionally wherein the second iblastoid media comprises an FGF component and further comprises an epidermal growth factor component, optionally about 50 nq/ml EGF.

16. (canceled)

17. The method of claim 1, wherein the hypoxic conditions comprise about 5%-10% CO₂, optionally about 6.8% CO₂, and about 1%-5% O₂, optionally about 5% O₂.

18. The method of claim 1, wherein the first iblastoid media and/or the second iblastoid media is replaced with fresh media after about 24 hours.

19. The method of claim 1, wherein the population of bovine naïve stem cells is derived from primed pluripotent stem cells or induced pluripotent stem cells, or wherein the population of bovine naïve stem cells are embryo-derived naïve stem cells, optionally derived from a preimplantation embryo or embryo-derived expanded pluripotent stem cells, optionally the population of bovine naïve stem cells are derived by a method comprising:
a) providing a bovine embryo comprising bovine naïve stem cell-like cells, optionally wherein the bovine embryo is Zona Pellucida (ZP)-free;
b) contacting the bovine embryo with an extracellular matrix (ECM)-coated substrate, wherein the ECM-coated substrate comprises a substrate comprising a negatively charged substrate surface adjacent to a positively charged biocompatible polymer layer, and a negatively charged ECM layer adjacent to the positively charged biocompatible polymer layer; and
c) culturing the bovine embryo in the presence of outgrowth medium to induce attachment of the bovine embryo to the ECM-coated substrate and outgrowth of an inner cell mass (ICM) comprising derived bovine naïve stem cells, thereby deriving a population of bovine naïve stem cells.

20. The method of claim 1, wherein the bovine naïve stem cells are genetically modified and/or genome edited cells.

21. The method of claim 1, further comprising performing testing for one or more biomarkers to determine one or more characteristics of the population of bovine naïve stem cells for one or more biomarkers, and optionally determining a score based on the determined characteristics, and optionally further comprising selecting the population of bovine naïve stem cells based on the one or more characteristics determined by testing for one or more biomarkers and/or the score.

22. (canceled)

23. The method of claim 1, further comprising performing testing for one or more biomarkers to determine one or more characteristics of the iblastoids, and optionally determining a score based on the determined characteristics, optionally further comprising selecting one or more of the iblastoids based on the one or more characteristics determined by testing for one or more biomarkers and/or the score.

24. (canceled)

25. (canceled)

26. A method of cattle breeding, the method comprising:
a) selecting at least one bovine parent from a plurality of animals;
b) obtaining a plurality of gametes from the at least one bovine parent;
c) creating at least one embryo from the plurality of gametes;
d) deriving a population of naïve stem cells from the at least one embryo;
e) preparing one or more iblastoids according to the method of claim 1; and
f) selecting the one or more iblastoids for breeding, optionally in vitro breeding, and/or for transfer to recipient female surrogates for producing offspring.

27.-38. (canceled)
